(19) United States
(12) Patent Application Publication
(10) Pub. No.: US 2006/0057611 A1
(43) Pub. Date: Mar. 16, 2006

(54) Log-linear amplification

(75) Inventors: H. Pin Kao, Fremont, CA (US); Kai Qin Lao, Pleasanton, CA (US); Robert Jones, Los Altos, CA (US)

Correspondence Address: DECHERT LLP, P.O. BOX 10004, PALO ALTO, CA, 94303, US

(73) Assignee: Applera Corporation, Foster City, CA (US)

(21) Appl. No.: 11/173,902

(22) Filed: Jun. 30, 2005

Related U.S. Application Data
(60) Provisional application No. 60/584,665, filed on Jun. 30, 2004.

(51) Int. Cl. C12Q 1/68; G06F 19/00; C12P 19/34

(52) U.S. Cl. 435/6; 435/91.2; 702/20

(57) ABSTRACT

The present disclosure provides methods and composition to detect or quantitate one or more target sequences in a reaction that couples a linear amplification reaction to an exponential amplification reaction.

This application claims benefit under 35 U.S.C. § 119(e) of U.S. Patent Application Ser. No. 60/584,665, filed Jun. 30, 2004, which is incorporated herein by reference in its entirety

### 1. FIELD

This disclosure relates generally to compositions, methods, kits and apparatuses for carrying out nucleic acid sequence amplification, and more specifically to compositions, methods, kits and apparatuses for detecting and/or quantitating polynucleotide sequences.

### 2. INTRODUCTION

The most common methods of detecting or quantitating target polynucleotide sequences are array based assays and quantitative polymerase chain reaction (PCR). In general, array based assays are less sensitive and less specific than quantitative PCR assays. But array based assays have a very high throughput capacity, because large numbers of assays can be run simultaneously and signal detection is relatively simple. Quantitative PCR assays are more sensitive and specific than array based assays, and have a higher dynamic range. But quantitative PCR continuously monitors product accumulation and therefore is relatively slow, requiring about two hours for completion. The reaction rate of quantitative PCR is further extended by the low throughput capacity of existing PCR machines.

There is, accordingly, a need in the art for methods of detecting and quantitating target polynucleotide sequences that combine the sensitivity, specificity and dynamic range of quantitative PCR with the high throughput capacity of array based assays.

### 3. SUMMARY

Disclosed herein are compositions and methods for asymmetrically amplifying one or more target polynucleotide sequences of unknown copy number via a reaction that couples an exponential phase that generates double-stranded amplicons with a linear phase that generates single-stranded amplicons. In some embodiments, the exponential phase terminates before reaching a plateau when a selected number of double-stranded amplicons are produced. In some embodiments, the cycle number at which the exponential phase terminates can be obtained from the number of linear amplicons that are produced. In some embodiments, the number of linear amplicons produced can be determined directly. In some embodiments, the number of linear amplicons produced can be determined using a reporter molecule that produces a detectable signal proportional to the number of linear amplicons. Once the number of linear amplicons produced at a selected time point can be determined, the time point at which the exponential phase terminates, and the copy number of the target polynucleotide sequence can be obtained therefrom.

In some embodiments, the exponential and linear phases can be coupled by the exponential amplicons being a template for linear amplification. Therefore, the exponential and linear phases can be PCR based assays. In some embodiments, the exponential and/or linear phases can be another type of amplification reaction, including but not limited to, ligase based reactions. In some embodiments, the exponential and linear phases can be coupled by the product of the exponential phase functioning as a primer for the linear phase.

In another aspect, the disclosure provides kits for performing the various embodiments of the disclosed methods. The composition of kits can vary with the type of exponential and amplification reaction employed, the number and types of target sequences and the method of coupling the exponential and linear phases. In some embodiments, kits can comprise exponential and linear amplification primers, and a reporter molecule suitable for providing a signal proportional to the number of linear amplicons. In some embodiments, kits can comprise ligation probes suitable for exponential and/or linear amplification.

In another aspect, the disclosure provides devices for implementing the various embodiments of the log-linear amplification methods. In some embodiments, a device comprises components for implementing and monitoring amplification reactions and can contain components, such as, a thermal module and an optical module comprising an excitation source and a detector. In some embodiments, the disclosed devices comprise a processor directed by readable memory. The readable memory comprises executable instructions to direct the processor to implement the log-linear amplification methods. In some embodiments, the processor can be directed to determine the copy number of a target sequence.

### 4. BRIEF DESCRIPTION OF THE DRAWINGS

The skilled artisan will understand that the drawings, described below, are for illustration purposes only. The drawings are not intended to limit the scope of the present disclosure in any way.

The skilled artisan will understand that the drawings, described below, are for illustration purposes only. The drawings are not intended to limit the scope of the present disclosure in any way.

FIG. 1 provides a graph illustrating the results of a conventional PCR A and an embodiment of a log-linear amplification B monitored in real-time. Cₜ is the cycle number at which a detectable signal is produced above background.

FIG. 2 provides a graph illustrating the results of an embodiment of real-time log-linear single-plex amplification reactions carried out with varying copy numbers of the same target sequence under the same conditions. The graph demonstrates that the slopes of the linear phase of the log-linear amplifications are substantially constant for each target sequence, regardless of their differing copy numbers. Cₜ1 and Cₜ2 are the cycle numbers or time points at which a detectable number of exponential amplicons are produced above background for target sequences 1 and 2, respectively. In this embodiment, F1 and F2 are the fluorescent signals measured, which are proportional to the linear amplicons produced by each reaction at the same cycle number or time point.

FIG. 3 provides a cartoon illustrating an embodiment of a log-linear amplification reaction, in which a 5′-nuclease probe is used to produce a detectable signal proportional to the number of linear amplicons generated.

FIG. 4 illustrates the results of an embodiment of a log-linear amplification carried out with 100 pg (10⁷ copies), 10 pg (10⁶ copies), 1 pg (10⁵ copies; 1,000 fg), 100 fg (10⁴ copies), 10 fg (10³ copies), 1 fg (10² copies; 1,000 ag), 100 ag (10¹ copies), 10 ag (1 copy), and 1 ag (10⁻¹ copies) of liver ATP5b cDNA target sequence. The results shown are corrected using an internal standard, FRET-ROX (“ROX”, Applied Biosystems, Foster City, Calif.). Panel A provides a graph illustrating the results of the log-linear amplifications monitored in real-time (Iₘ v. cycle number). Panel B provides a graph illustrating a linear regression analysis for cycle numbers 1, 50 and 80.

FIG. 5 illustrates the results of a replicate of the experiment shown in FIG. 4. Panel A provides a graph illustrating the results of the log-linear amplifications monitored in real-time (ΔIₘ v. cycle number). ΔIₘ=Iₘ minus the baseline or background fluorescence intensity measured before the reaction produces a detectable signal (e.g., cycle nos. about 3 to about 14). Panel B provides a graph illustrating a linear regression analysis for cycle numbers 1, 50 and 80.

FIG. 6 illustrates the results of an embodiment of the log-linear amplification carried out with 16 pg (1.6×10⁶ copies), 8 pg (8×10⁵ copies), 4 pg (4×10⁵ copies), 2 pg (2×10⁵ copies), 1 pg (1×10⁵ copies), 0.5 pg (5×10⁴ copies; 500 fg), 250 fg (2.5×10⁴ copies), 125 fg (1.25×10⁴ copies), 62.5 fg (6.25×10³ copies), and 31 fg (3.1×10³ copies) of a liver ATP5b cDNA target sequence. Panel A provides a graph illustrating the results of the log-linear amplifications monitored in real-time (Iₘ v. cycle number). Panel B provides a graph illustrating the results of the log-linear amplifications monitored in real-time (ΔIₘ v. cycle number).

FIG. 7 provides a cartoon illustrating an embodiment of a log-linear amplification reaction as applied to gene expression analysis in which an exponential phase PCR amplification is coupled to a linear phase ligation amplification.

FIG. 8 provides a cartoon illustrating an embodiment of a log-linear amplification in which the exponential phase produces a sequence that functions as a linear primer. Panel A provides a cartoon illustrating one embodiment of an exponential amplification reaction in which a sequence is generated by cleavage of a flap probe. Panel B provides a cartoon illustrating one embodiment in which the sequence generated by the embodiment illustrated in Panel A may be used in an embodiment of a coupled linear amplification reaction.

FIG. 9, Panel A provides a graph illustrating of mean Cₜ value vs. Iₘ obtained by log-linear amplification of ATP5b cDNA performed in triplicate under the embodiment described in FIG. 4 and Example 1. Panel B provides a graph illustrating the results of one embodiment in which the mean Cₜ value vs. ΔIₘ obtained by log-linear amplification of ATP5b cDNA performed in triplicate under the embodiment described in FIG. 4 and Example 1.

FIG. 10, Panel A provides a graph illustrating the linear regression analysis for cycle number 50 for the log-linear amplification shown in FIG. 9A. Panel B provides a graph illustrating the linear regression analysis for cycle number 50 for the log-linear amplification shown in FIG. 9B.

FIG. 11, Panel A provides a graph illustrating the linear regression analysis for cycle number 50 for the log-linear amplification data shown in FIG. 6A. Panel B provides a graph illustrating the linear regression analysis for cycle number 50 for the log-linear amplification shown in FIG. 6B

FIG. 12 provides a flow diagram of an embodiment of a log-linear amplification.

FIG. 13 provides a schematic illustrating one embodiment of an instrument suitable for conducting log-linear amplification reactions having a thermal cycling system comprising reaction module 20 linked to thermal control module 10. Reaction module 20 is optically linked 30 to optical module or optical head 40 having excitation source 50 and detector 60. The optical module and thermal control module are operably linked to processor or computer 70 directed by computer readable memory 80. The output of processor 70 is directed to output device 90.

### 5. DETAILED DESCRIPTION

It is to be understood that both the foregoing general description, including the drawings, and the following detailed description are exemplary and explanatory only and are not restrictive of this disclosure. In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

This disclosure provides compositions, methods, kits and instrumentation for detecting or quantitating polynucleotide sequences.

As discussed in the Summary section, the disclosed methods asymmetrically amplify a target polynucleotide sequence of unknown copy number via an amplification reaction that couples an exponential phase that generates a double-stranded “exponential” amplification product (“amplicon”) with a linear phase that generates a single-stranded “linear” amplicon. In general, the phases are coupled by the product of the exponential phase serving as a reactant in the linear phase. Therefore, in some embodiments, the exponential phase produces amplicons that serve as templates for the linear phase. In some embodiments, the exponential phase produces a sequence that functions as a primer for the linear phase. The conditions of the amplification reaction are adjusted so that the exponential phase of the reaction terminates prior to reaching a plateau. The number of copies of the target polynucleotide sequence (“copy number”) in the original sample can be determined from the rate of accumulation of linear amplicons. In some embodiments, the reaction conditions are adjusted such that a predetermined number of exponential amplicons are produced. Since the rate of accumulation of linear amplicons depends upon the number of exponential amplicons generated, the copy number of the target sequence present in the sample can be obtained by determining the amount of linear amplicon generated at a single time point.

Asymmetric amplifications in which the reaction conditions are designed to terminate the exponential phase before it reaches a plateau are referred to herein as “log-linear” amplifications. Such log-linear amplifications can be carried out in a “single-plex” mode in which a single target sequence of interest is amplified or in a “multiplex” mode in which a plurality of different target sequences are amplified in a single amplification reaction. The copy numbers of the different target sequences present in the amplified sample can be determined from the rate of accumulation of their corresponding linear amplicons. In some embodiments of multiplex log-linear amplifications, the reaction conditions are designed so that each exponential amplification generates a selected equivalent number of exponential amplicons. Because the linear phases utilize a product of the exponential phase, the rates of accumulation of the corresponding linear amplicons is proportional to the number of double-stranded exponential amplicons generated. Since the numbers of exponential amplicons generated for each target sequence are essentially equivalent, the rates of accumulation of the various linear amplicons are substantially constant. By amplifying the target sequences in this manner, the copy numbers of the different target sequences present in the original sample can be obtained from a single measurement, as will be described in more detail below.

The log-linear amplifications described herein couple an exponential phase amplification that generates a double-stranded amplification product or amplicon with a linear phase amplification that generates a single-stranded linear amplification product or amplicon 100. The two phases are coupled in the sense that the product of the exponential phase of the amplification serve as template for the linear phase of the amplification. As will be recognized by skilled artisans, methods for exponentially amplifying and linearly amplifying polynucleotide sequences are known in the art. For example, methods of exponentially amplifying polynucleotide sequences of interest via the polymerase chain reaction (PCR) are described in, e.g., U.S. Pat. Nos. 4,683,195, 4,683,202, 4,800,159, 4,965,188, 5,075,216, 5,176,995, 5,386,022, 5,333,675, 5,656,493, 6,040,166, 6,197,563, 6,514,736, and EP-A-0200362 and EP-A-0201184. Methods of exponentially amplifying polynucleotide sequences via the ligase chain reaction are described in, e.g., U.S. Pat. Nos. 5,427,930, 5,516,663, 5,686,272, 5,869,252 and EP-A-320308. Methods of linearly amplifying polynucleotides sequences of interest via polymerization reactions are described in, e.g., U.S. Pat. Nos. 5,066,584, 5,891,625 and WO 93/25706. Methods of linearly amplifying polynucleotide sequences of interest via ligation reactions are described in, e.g., U.S. Pat. Nos. 5,185,243 and 5,679,524. All of these various methods can be utilized in various combinations to amplify polynucleotides via the log-linear methods described herein.

In some embodiments, the exponential phase of the amplification is carried out using principles and reagents for amplifying DNA polynucleotides via the polymerase chain reaction (“PCR”) or, in cases where the sample contains an RNA polynucleotide, via the reverse-transcription polymerase chain reaction (“RT-PCR”).

In such embodiments, exponential phase or double-stranded amplicons are produced by thermocycling a target polynucleotide sequence in the presence of two primers (“forward” and “reverse exponential primers”), a polymerase (e.g., a thermostable polymerase), and a mixture of 3′-deoxyribonucleotide triphosphates (“dNTPs”) suitable for DNA synthesis. The skilled artisan will appreciate that an RNA target polynucleotide is reverse transcribed into a cDNA, which is exponentially amplified. Thus, during each thermocycle, the primers anneal to the DNA or cDNA target sequence at sites removed from one another and in orientations such that the extension product of one primer (e.g., the forward primer) when separated from its complement, can hybridize to the extension product of the other primer (e.g., the reverse primer). Thus, amplification products generated by the exponential phase (“exponential amplicons”) are discrete double-stranded DNAs having: (i) a first strand which includes, from 5′ to 3′, the sequence of the forward primer, the complement of the target sequence of interest, and a sequence complementary to the reverse primer, and (ii) a second strand which is complementary to the first strand. In successive thermocycles, the double-stranded amplicons produced in earlier cycles serve as templates for double-stranded amplicon synthesis. Therefore, with each successive cycle, double-stranded amplicons accumulate exponentially by a theoretical factor of two. Multiplex exponential phase amplification can be carried out using a plurality of sets of primers each of which amplifies a different target sequence.

In a log-linear amplification, the reaction conditions are established so that the exponential phase amplification terminates before double-stranded amplicon synthesis plateaus. This is in contrast to a conventional PCR, which plateaus when one or more reagents are consumed or the hybridization of the first and second strands of the double-stranded amplicons to each other out competes the hybridization of the amplification primers to the individual amplicon strands. Therefore, in a conventional PCR, DNA synthesis (and consequent accumulation of double-stranded amplicons) may terminate even though primers, template, polymerase and dNTPs are still present.

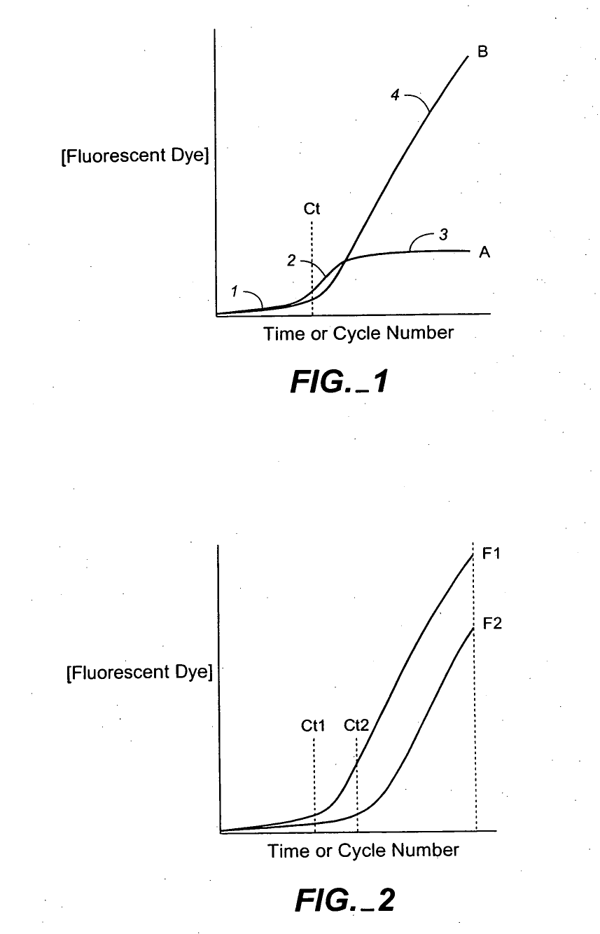

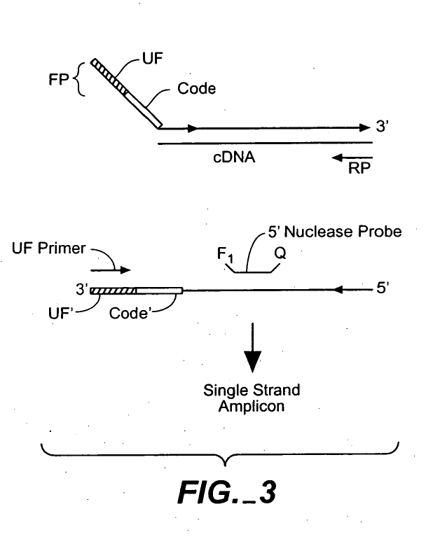

To illustrate the time points at which the exponential phase of a log-linear amplification reaction may terminate, an embodiment of a log-linear amplification employing PCR-based exponential and linear phases is compared to a conventional PCR in FIG. 1. In FIG. 1, the conventional PCR amplification is trace A; the log-linear amplification is trace B. Both amplifications were monitored in real-time using a sequence specific 5′-nuclease probe bearing a reporter fluorophore at one end and a quencher moiety at the other end (TaqMan® probe). The conventional PCR can be roughly divided into three distinct regions or phases: an initial phase 1, and exponential phase 2 and a plateau phase 3. In the initial phase of the PCR, the number of double-stranded amplicons does not detectably increase and is below the threshold level of the sensitivity of the detection system being used. Thus, in this initial phase 1, the accumulation of double-stranded amplicons goes undetected. When the threshold number of double-stranded amplicons capable of being detected is present, i.e., when the Cₜ (“cycle threshold”) of the reaction is reached, a conventional PCR enters an exponential phase 2 during which the number of double-stranded amplicons detectably increases exponentially with each successive thermocycle. As PCR reagents become limiting, or the amplification primers are no longer able to productively anneal to the individual strands of the double-stranded amplicons, DNA synthesis slows and eventually stops or plateaus 3. In contrast, a log-linear amplification B continues to produce single-stranded amplicons 4 and a detectable signal at cycle numbers where a conventional PCR plateaus.

In contrast to a conventional PCR, the exponential phase of a log-linear amplification reaction is designed to terminate before it reaches a plateau. The point prior to plateau at which the exponential phase of a log-linear amplification terminates is not critical for success. The exponential phase may be designed to terminate before a detectable number of double-stranded amplicons are produced (i.e., before the Cₜ), at or about the cycle number when a detectable number of double-stranded amplicons are produced (i.e., at or about the Cₜ), or after a detectable number of double-stranded amplicons are produced, but before the exponential phase plateaus (i.e., during phase 2 but before phase 3).

In some embodiments, the exponential phase is designed to terminate when a pre-selected number of double-stranded amplicons are produced for each target sequence being amplified 110. In such embodiments, a substantially uniform number of double-stranded amplicons are produced from each target sequence, regardless of the number of copies of each sequence present in the original sample.

Selecting the number of double-stranded amplicons to be produced by the exponential phase of a log-linear reaction and designing reaction conditions suitable for yielding the desired results is within the abilities of the skilled artisan. Non-limiting examples of factors to be considered include the quantity of each target polynucleotide sequence, the relative amount of each target polynucleotide sequence, the number of different target polynucleotide sequences to be amplified in a single reaction (i.e., multiplex or single-plex), the sensitivity of the detection system used to measure accumulation of amplification products, and the degree of accuracy desired. Therefore, in various exemplary embodiments, the number of double-stranded amplicons produced by the exponential phase may be at or about the threshold number of double-stranded amplicons that is capable of being detected (i.e., Cₜ), greater than the number of double-stranded amplicons that is capable of being detected, or below the number of double-stranded amplicons that is capable of being detected. By “the number of double-stranded amplicons that is capable of being detected” is meant the minimum number of double-stranded amplicons that are detectable by the system used to measure the signal produced by the linear phase reaction. The skilled artisan is aware that the exponential phase reaction conditions may be established using a different detection system than that used for the linear phase reaction. The detection system used to establish the exponential phase conditions may be, for example, more sensitive than the system utilized in the log-linear reaction. Therefore, it is possible to establish exponential phase conditions that terminate exponential amplification when a selected number of double-stranded amplicons is produced that is below the threshold level required for detection. The skilled artisan is aware, that in some embodiments, the number of double-stranded amplicons produced can be obtained from the starting concentrations of the exponential amplification primers.

Conditions for terminating the exponential phase can be determined empirically and may depend upon the type of reaction used for the exponential phase. For example, various exponential phase parameters, such as the concentration and relative amounts of the exponential amplification primers, may be systematically varied and each set of conditions may be monitored in real-time as known in the art. Once exponential phase conditions are established and optimized that are suitable for the detection system selected by the skilled artisan, the conditions are generally applicable to virtually any target sequence from any source. For example, in this embodiment general primer concentrations can be established that can be applied to virtually any target sequence, as further described below. However, in other embodiments conditions for terminating the exponential phase, e.g., primer concentration, can be tailored to specific target sequences. As described above, selecting the number of double-stranded amplicons to be produced and the exponential phase conditions for producing the selected number of double-stranded amplicons are within the abilities of the skilled artisan. However, in general, a target sequence will be exponentially amplified for at least about 2-5 thermocycles. In other embodiments, a target sequence is exponentially amplified for at least about 6-10 thermocycles. In yet other embodiments, a target sequence is exponentially amplified at least about 11-20 thermocycles or more. The length of the exponential and linear amplicon may vary widely. In some embodiments, the length of an amplicon may be at least about 10 base pairs, at least about 100 base pairs, at least about 500 base pairs, or at least about 1000 base pairs or greater. In some embodiments, the exponential amplicon is about 75 to about 200 base pairs.

As used herein, “Cₑ” is the time point or cycle number at which the selected number of double-stranded amplicons is produced for each target polynucleotide that is exponentially amplified 110. Therefore, Cₑ may also be defined as the time point or cycle number at which the exponential phase of the log-linear amplification terminates. Cₑ is a logarithmic function based on the theoretical doubling of the double-stranded amplicons with each successive round of exponential-phase amplification. In addition, Cₑ is directly proportional to the pre-selected number of double-stranded amplicons generated and inversely proportional to the number of copies of the target sequence present in the sample being amplified (“copy number”). Therefore, Cₑ may be defined as set forth in Equation (1):

$${C}_{e} = {log}_{2} ( \frac{[ {A}_{e} ]}{[ {T}_{n} ]} ) \qquad ( 1 )$$

where, [Aₑ] is the number of double-stranded amplicons produced by the exponential phase of the amplification at cycle Cₑ; and [Tₙ] is the number of target sequences (copy number) present in the amplification reaction prior to amplification.

Solving Equation (1) for [Tₙ] yields Equation (2):
[Tₙ]=([Aₑ])(2^{-Ce}) (2).

According to Equation (2), the number of target sequences prior to amplification may be determined if the number of double-stranded amplicons produced at cycle Cₑ is known. As described above, in some embodiments, the number of double-stranded amplicons produced from each target sequence is known because the exponential phase is designed to produce an equivalent, selected number of double-stranded amplicons from each target sequence. Therefore, to calculate the number of target sequences prior to amplification, Cₑ can be determined.

In a log-linear amplification reaction, Cₑ can be determined based upon the rate at which the amplification products of the linear phase of the log-linear amplification accumulate. Like the exponential phase, the linear phase can be accomplished using any technique capable of linearly amplifying the exponential amplicons. Therefore, in some embodiments, the linear phase reaction may involve thermocycling using a thermostable enzyme, such as, a polymerase or ligase. In other embodiments, as described in more detail below, thermocycling is not used.

In some embodiments, the linear phase reaction comprises hybridizing one primer (“linear primer”) to one strand of the double-stranded amplicons in the presence of a DNA polymerase and a mixture of dNTPs suitable for DNA synthesis. The linear phase of the reaction is carried out under conditions in which the rate of accumulation of the linear amplicons is proportional to the number of double-stranded amplicons present in the amplification reaction. For embodiments employing linear primer-initiated synthesis, this can be accomplished by utilizing excess linear primer. The amount of excess linear primer is not critical for success, provided that the concentration supports the desired number of rounds of linear amplification. Suitable linear primer:exponential amplicon concentration ratios are discussed in more detail in a later section. Differences observed between log-linear amplifications of various target sequences will therefore relate to target sequence copy number, the cycle number, Cₑ, at which the selected number of double-stranded amplicons is produced, the cycle number, C_{d}, at which the amount of single-stranded amplicons is determined and detectable signal is measured. The first-order kinetic relationship of these parameters is shown in Equation (3):
Aₗ=(C_{d}−Cₑ)L (3)
where, Aₗ is the amount of linear amplicon measured, directly without use of a reporter molecule or indirectly via a reporter molecule 120, above background at cycle number C_{d}. C_{d} is the Aₗ cycle number 130; and L is the rate of the accumulation of linear amplicons (ΔAₗ/Δcycle number). As described in more detail below, in some embodiments, Aₗ can be measured directly, for example, by capillary electrophoresis (CE) in which one or more aliquots of the reaction are sampled. In other embodiments, Aₗ is measured indirectly by using a reporter molecule, such as a nucleic acid binding agent that produces a detectable signal in proportion to the linear amplicons. Thus, in some embodiments, the detectable signal can be measured by a homogenous assay system as is known in the art. Accordingly, in some embodiments, a detectable signal, Iₘ, can be measured as ratio of a detected signal/reference signal (I_{detected}(I_{d})/I_{reference} (Iᵣ)). In some embodiments, a detectable signal, ΔIₘ, can be measured as Iₘ−I_{baseline}(I_{b}).

Solving Equation (3) for Cₑ yields Equation (4):

$${C}_{e} = {C}_{d} - ( \frac{{A}_{l}}{L} ) . \qquad ( 4 )$$

According to Equations 2 and 4, by establishing exponential phase reaction conditions that generate substantially uniform numbers of double-stranded amplicons for each target sequence being amplified, and linearly amplifying the double-stranded amplicons at a substantially constant rate, the Cₑ value and, therefore, Tₙ may be determined by measuring the amount of linear amplicon generated at one cycle number or time point. In an alternative embodiment, L may be monitored as a function of time and determined empirically. By “function of time” herein is meant that the amount of linear amplicon generated is measured at one or more specific time points or cycle numbers. However, in an embodiment in which substantially equivalent numbers of exponential amplicons are generated, once the log-linear reaction conditions are established, and Aₑ and L are substantially constant, monitoring the rate of linear amplification is advantageously avoided and a single time point measurement of the amount of linear amplicon generated may be used to determine Cₑ using Equation (4) and Tₙ using Equation (2) 140. Alternatively, Tₙ can be determined by combining Equations (2) and (4), to yield Equation (5):

$$[ {T}_{n} ] = ( [ {A}_{e} ] ) {2}^{- ( {C}_{d} - ( \frac{{A}_{l}}{L} ) )} . \qquad ( 5 )$$

As will be appreciated by skilled artisans, target polynucleotides comprising one or more target sequences suitable for log-linear amplification may be either DNA (e.g., cDNA, genomic DNA or extrachromosomal DNA) or RNA (e.g., mRNA, rRNA or genomic RNA) in nature, and may be derived or obtained from virtually any sample or source, wherein the sample may optionally be scarce or of a limited quantity. For example, the sample may be one or a few cells collected from a crime scene or a small amount of tissue collected via biopsy. In other embodiments, the target polynucleotide may be a synthetic polynucleotide comprising nucleotide analogs or mimics, as described below, produced for purposes, such as, diagnosis, testing, or treatment. Therefore, by “suitable for log-linear amplification” herein is meant the target polynucleotide comprises at least one sequence that is capable of being amplified by the disclosed methods.

In various non-limiting examples, the target polynucleotide may be single or double-stranded or a combination thereof, linear or circular, a chromosome or a gene or a portion or fragment thereof, a regulatory polynucleotide, a restriction fragment from, for example, a plasmid or chromosomal DNA, genomic DNA, mitochondrial DNA, DNA from a construct or library of constructs (e.g., from a YAC, BAC or PAC library), RNA (e.g., mRNA, rRNA or vRNA) or a cDNA or a cDNA library. As known in the art, a cDNA is a single- or double-stranded DNA produced by reverse transcription of an RNA template. Therefore, in some embodiments, in addition to the amplification primers and DNA polymerase include a reverse transcriptase and one or more primers suitable for reverse transcribing an RNA template into a cDNA. Reactions, reagents and conditions for carrying out such “RT” reactions are known in the art (see, e.g., Blain et al., 1993, J. Biol. Chem. 5:23585-23592; Blain et al., 1995, J. Virol. 69:4440-4452; PCR Essential Techniques 61-63, 80-81, (Burke, ed., J. Wiley & Sons 1996); Gubler et al., 1983, Gene 25:263-269; Gubler, 1987, Methods Enzymol., 152:330-335; Sellner et al., 1994, J. Virol. Method. 49:47-58; Okayama et al., 1982, Mol. Cell. Biol. 2:161-170; and U.S. Pat. Nos. 5,310,652, 5,322,770, and 6,300,073, these disclosures of which are incorporated herein by reference.

The target polynucleotide may include a single polynucleotide, from which one or more different target sequences of interest may be amplified, or it may include a plurality of different polynucleotides, from which one or more different target sequences of interest may be amplified. As will be recognized by skilled artisans, the sample or target polynucleotide may also include one or more polynucleotides comprising sequences that are not amplified in the log-linear reaction.

In some embodiments of log-linear amplifications, highly complex mixtures of target sequences from highly complex mixtures of polynucleotides are amplified in either a single-plex or multiplex format. Indeed, many embodiments are suitable for multiplex log-linear amplification of target sequences from tens, hundreds, thousands, hundreds of thousands or even millions of polynucleotides. In some embodiments, multiplex amplification methods can be used to amplify pluralities of target sequences from samples comprising cDNA libraries or total mRNA isolated or derived from biological samples, such as tissues and/or cells, wherein the cDNA or, alternatively, mRNA libraries may be quite large. For example, cDNA libraries or mRNA libraries constructed from several organisms or from several different types of tissues or organs can be amplified according to the methods described herein.

As the skilled artisan will appreciate, multiple sets of primers and/or probes and/or reporter molecules are utilized for each target sequence to be analyzed by multiplex log-linear amplification reactions. For example, in multiplex embodiments utilizing reporter molecules to analyze each target sequence, each reporter molecule can produce a signal that is distinguishable from other reporter molecules. Therefore, in these embodiments, the number of target sequences analyzed in a multiplex format can be determined, at least in part, by the number and type of reporter molecules that may be discriminated. For example, in the embodiment, in which 5′-nuclease (e.g., TaqMan®) probes are utilized as the reporter molecule about 2 to about 7 target sequences are analyzed in a multiplex reaction. However, in other embodiments in which “flap” probes are utilized as a reporter molecules about 2 to about 1,000 target sequences and in some embodiments to about 7000 target sequences or more are analyzed in a multiplex reaction (see, e.g., U.S. Patent Application Ser. Nos. 60/584,621, 60/584,596, 60/584,643, each filed Jun. 30, 2004).

The amount of target polynucleotide(s) included in a log-linear amplification reaction can vary widely. In many embodiments, amounts suitable for a conventional PCR and/or RT-PCR may be used. For example, the target polynucleotide(s) may be from a single cell, from tens of cells, from hundreds of cells or even more, as is well known in the art. For many embodiments, including embodiments in which the target polynucleotide is a complex cDNA library (or derived therefrom by RT of mRNA), the total amount of target polynucleotide in a log-linear amplification may range from about 1 pg to about 100 ng. For other embodiments, including embodiments in which the target polynucleotide(s) is obtained from a single cell, the total amount of target polynucleotide(s) included in a log-linear amplification may range from about about 10 ag to about about 100 pg. In some embodiments, the total amount of target polynucleotide(s) may range from about 1 copy to about 10⁷ copies.

In some embodiments, preparation of the target polynucleotide(s) for analysis may not be required. In some embodiments, the target polynucleotide(s) may be prepared for log-linear amplification using conventional sample preparation techniques suitable for the type of amplification reaction to be used. For example, target polynucleotides may be isolated from their source via differential extraction, centrifugation, chromatography, precipitation, electrophoresis, as is well-known in the art. Alternatively, the target sequence(s) may be amplified directly from samples, including but not limited to, cells or from lysates of tissues or cells comprising the target polynucleotide(s).

The number of target sequences that can be amplified by a log-linear amplification is influenced in large part by the number of different amplification primers used during the log-linear amplification and the number of different methods used to detect or discriminate the amplification products. In some embodiments, at least two amplification primers are used for log-linear amplification of a target sequence. In other embodiments, at least three amplification primers are used. “Primer” herein refers to a polynucleotide capable of hybridizing or annealing to a template polynucleotide to form a substrate for a polymerase (e.g., DNA-dependent DNA polymerases, RNA-dependent DNA polymerase (reverse transcriptase)). Therefore, in various embodiments, a primer can be an amplification primer and/or a reverse transcription primer. “Annealing” or “hybridizing” refer to base-pairing interactions of one nucleobase polymer with another that results in the formation of a double-stranded structure. In some embodiments, annealing occurs via Watson-Crick base-pairing interactions, but may be mediated by other hydrogen-bonding interactions, such as Hoogsteen base pairing. When a primer is hybridized to its template, a polymerase initiates synthesis of a nascent polynucleotide strand in a template directed manner at the 3′ terminus of the primer.

In various embodiments, an amplification primer is an “exponential primer” and/or a “linear primer.” By “exponential primer” and “exponential amplification primer” herein are meant a primer suitable for exponential amplification of a polynucleotide sequence. In exponential target sequence amplification, the product of each amplification cycle is an amplicon that is a suitable template for subsequent amplification cycles. Therefore, as known in the art, exponential amplification generally utilizes at least two exponential primers. For example, the exponential amplification of a target sequence by PCR generally utilizes a pair of “forward” and “reverse” primers. Therefore, the skilled artisan is aware that the suitability of a primer for exponential amplification depends, in part, on the presence of a second suitable primer. The forward and reverse primers hybridize to a target sequence in opposite orientations to produce complementary DNA strands to form double-stranded amplicons that serve as templates for further rounds of amplification. By “linear primer” and “linear amplification primer” herein are meant a primer suitable to linearly amplify a polynucleotide sequence. In linear target sequence amplification, the product of each amplification cycle is not suitable for subsequent amplification cycles. For example, the linear amplification of a target sequence generally produces a single-stranded amplicon that does not hybridize to the linear primer and, therefore, it is not a suitable template for subsequent amplification cycles. As a result, linear amplicons accumulate at a rate proportional to the number of templates.

The amplification primers may be target sequence-specific or may be designed to hybridize to sequences that flank a target sequence to be amplified. Thus, the actual nucleotide sequences of each primer may depend upon the target sequence and target polynucleotide, which will be apparent to those of skill in the art. Methods for designing primers suitable for amplifying target sequences of interest are well-known (see, e.g., Dieffenbach et al., General Concepts for PCR Primer Design, in PCR Primer, A Laboratory Manual, Dieffenbach, C. W, and Dveksler, G. S., Ed., Cold Spring Harbor Laboratory Press, New York, 1995, 133-155; Innis, M. A. et al. Optimization of PCRs, in PCR protocols, A Guide to Methods and Applications, Innis, M. A., Gelfand, D. H., Sninsky, J. J., and White, T. J., Ed., CRC Press, London, 1994, 5-11; Sharrocks, et al. The design of primers for PCR, in PCR Technology, Current Innovations, Griffin, H. G., and Griffin, A. M, Ed., CRC Press, London, 1994, 5-11; Suggs et al., Using Purified Genes, in ICN-UCLA Symp. Developmental Biology, Vol. 23, Brown, D. D. Ed., Academic Press, New York, 1981, 683; Kwok et al. Effects of primer-template mismatches on the polymerase chain reaction: Human Immunodeficiency Virus 1 model studies. Nucleic Acids Res. 18:999-1005, 1990; Compton T (1990). Degenerate primers for DNA amplification. pp. 39-45 in: PCR Protocols (Innis, Gelfand, Sninsky and White, eds.); Academic Press, NY; Fuqua et al. (1990). BioTechniques 9 (2):206-211; Gelfand et al., 1990, Thermostable DNA polymerases. pp. 129-141 in: PCR Protocols (Innis, Gelfand, Sninsky and White, eds.); Academic Press, NY; Innis et al., 1990, Optimization of PCRs. pp. 3-12 in: PCR Protocols (Innis, Gelfand, Sninsky and White, eds.); Academic Press, NY; Krawetz et al., 1989, Nucleic Acids Research 17 (2):819; Rybicki et al., 1990, Journal of General Virology 71:2519-2526; Rychlik et al., 1990, Nucleic Acids Research 18 (21):6409-6412; Sarkar et al., 1990, Nucleic Acids Research 18 (24):7465; Smith et al., 1990, 9/90 (5):16-17; Thweatt et al. 1990, Analytical Biochemistry 190:314-316; Wu et al., 1991, DNA and Cell Biology 10 (3):233-238; Yap et al., 1991, Nucleic Acids Research 19 (7):1713.

Generally, each amplification primer should be sufficiently long to prime template-directed synthesis under the conditions of the log-linear reaction. The exact lengths of the primers may depend on many factors, including but not limited to, the desired hybridization temperature between the primers and template polynucleotides, the complexity of the different target polynucleotide sequences to be amplified, the salt concentration, ionic strength, pH and other buffer conditions, and the sequences of the primers and templates. The ability to select lengths and sequences of primers suitable for particular applications is within the capabilities of ordinarily skilled artisans (see, e.g., Sambrook et al. Molecular Cloning: A Laboratory Manual 9.50-9.51, 11.46, 11.50 (2d. ed., Cold Spring Harbor Laboratory Press); Sambrook et al., Molecular Cloning: A Laboratory Manual 10.1-10.10 (3d. ed. Cold Spring Harbor Laboratory Press)). In some embodiments, the primers contain from about 15 to about 35 nucleotides that are suitable for hybridizing to a target sequence and form a substrate suitable for DNA synthesis, although the primers may contain more or fewer nucleotides. Shorter primers generally require lower temperatures to form sufficiently stable hybrid complexes with target sequences. The capability of polynucleotides to anneal can be determined by the melting temperature (“Tₘ”) of the hybrid complex. Tₘ is the temperature at which 50% of a polynucleotide strand and its perfect complement form a double-stranded polynucleotide. Therefore, the Tₘ for a selected polynucleotide varies with factors that influence or affect hybridization. In some embodiments, in which thermocycling occurs, the amplification primers can be designed to have a melting temperature (“Tₘ”) in the range of about 60-75° C. Melting temperatures in this range tend to insure that the primers remain annealed or hybridized to the target polynucleotide at the initiation of primer extension. The actual temperature used for a primer extension reaction may depend upon, among other factors, for example, the concentration of the primers which are used in the log-linear reaction and the types of probes, as described below, used for detecting single-stranded amplicons and the amplifying agent. For amplifications carried out with a thermostable polymerase such as Taq DNA polymerase, in exemplary embodiments the amplification primers can be designed to have a Tₘ in the range of about 60 to about 78° C. or from about 55 to about 70° C. The melting temperatures of the different amplification primers can be different; however, in an alternative embodiment they should all be approximately the same, i.e., the Tₘ of each amplification primer can be within a range of about 5° C. or less. The Tₘs of various primers can be determined empirically utilizing melting techniques that are well-known in the art (see, e.g., Sambrook et al. Molecular Cloning: A Laboratory Manual 11.55-11.57 (2d. ed., Cold Spring Harbor Laboratory Press)). Alternatively, the Tₘ of a primer can be calculated. Numerous references and aids for calculating Tₘs of primers are available in the art and include, by way of example and not limitation, Baldino et al. Methods Enzymology. 168:761-777; Bolton et al., 1962, Proc. Natl. Acad. Sci. USA 48:1390; Bresslauer et al., 1986, Proc. Natl. Acad. Sci. USA 83:8893-8897; Freier et al., 1986, Proc. Natl. Acad. Sci. USA 83:9373-9377; Kierzek et al., Biochemistry 25:7840-7846; Rychlik et al., 1990, Nucleic Acids Res. 18:6409-6412 (erratum, 1991, Nucleic Acids Res. 19:698); Rychlik. J NIH Res. 6:78; Sambrook et al. Molecular Cloning: A Laboratory Manual 9.50-9.51, 11.46-11.49 (2d. ed., Cold Spring Harbor Laboratory Press); Sambrook et al., Molecular Cloning: A Laboratory Manual 10.1-10.10 (3d. ed. Cold Spring Harbor Laboratory Press)); Suggs et al., 1981, In Developmental Biology Using Purified Genes (Brown et al., eds.), pp. 683-693, Academic Press; Wetmur, 1991, Crit. Rev. Biochem. Mol. Biol. 26:227-259, which disclosures are incorporated by reference. Any of these methods can be used to determine a Tₘ of a primer.

As the skilled artisan will appreciate, in general, the relative stability and therefore, the Tₘs, of RNA:RNA, RNA:DNA, and DNA:DNA hybrids having identical sequences for each strand may differ. In general, RNA:RNA hybrids are the most stable (highest relative Tₘ) and DNA:DNA hybrids are the least stable (lowest relative Tₘ). Accordingly, in some embodiments, another factor to consider, in addition to those described above, when designing a primer is the structure of the primer and target polynucleotide. For example, in the embodiment in which an RNA polynucleotide is reverse transcribed to produce a cDNA, the determination of the suitability of a DNA primer for the reverse transcription reaction should include the effect of the RNA polynucleotide on the Tₘ of the primer. Although the Tₘs of various hybrids may be determined empirically, as described above, examples of methods of calculating the Tₘ of various hybrids are found at Sambrook et al. Molecular Cloning: A Laboratory Manual 9.51 (2d. ed., Cold Spring Harbor Laboratory Press).

Like amplification primers for conventional PCR or RT-PCR, the sequences of amplification primers useful for log-linear amplification are designed to be substantially complementary to regions of the target polynucleotides. By “substantially complementary” herein is meant that the sequences of the primers include enough complementarity to hybridize to the target polynucleotides at the concentration and under the temperature and conditions employed in the log-linear amplification reaction and to be extended by the DNA polymerase.

Although in some embodiments the sequences of the primers may be completely complementary to a target polynucleotide, in other embodiments it may be desirable to include one or more nucleotides of mismatch or non-complementarity, as is well known in the art. By “regions of mismatch” and “non-complementarity” are meant a least one nucleotide of a polynucleotide sequence that is not suitable for base-pairing with another polynucleotide sequence. Therefore, the term “region of mismatch” is used when comparing sequences, such as, a primer sequence and another primer sequence; a primer sequence and a target sequence; a probe sequence and a target sequence; a primer sequence and an amplicon sequence; and the like. Therefore, a “region of mismatch” includes a “region of sequence diversity.” In some embodiments, a primer sequence that is a region of mismatch in comparison to a target sequence is substantially unique to that primer. In other embodiments, a primer sequence that is a region of mismatch in comparison to a target sequence also occurs in other primers or probes. Therefore, in some embodiments, a region of mismatch between a primer and a target sequence is a code sequence. By “code sequence” is meant a primer sequence of continuous nucleotides that are not substantially complementary to a target sequence and is substantially unique to that primer. By “substantially unique” is meant the sequence is suitable to identify or distinguish the primer and the amplification products of the primer from other primers and other amplification products. Primers and methods for amplifying sequences to include such code sequence are known in the art (see, e.g., U.S. Pat. Nos. 5,314,809, 5,853,989, 6,090,552, 6,355,431 and, the disclosures of which are incorporated by reference).

In some embodiments, a region of mismatch between a primer and a target sequence is a sequence that is shared by more than one primer sequence. In various exemplary embodiments, a “shared sequence” may be common to each forward primer, each reverse primer, or each linear primer. Thus, by “forward universal sequence” and “reverse universal sequence” are meant a primer sequence of continuous nucleotides that is a region of diversity in comparison to a target sequence that is shared by each forward or reverse primer, respectively, in a log-linear amplification reaction (see, e.g., U.S. Pat. Nos. 5,882,856, 6,617,138, 6,630,329, 6,635,419, 6,670,130, 6,670,161 and Weighardt et al., 1993, PCR Methods and App. 3:77). Determining the number, type, length and composition of regions of mismatch and their position within a primer or probe and their distribution or commonality among the nucleic acids of a log-linear reaction are within the capabilities of the ordinary skilled artisan. Generally, regions of mismatch are designed to perform a user-selected function when incorporated into exponential or linear amplicons. The incorporated sequences provide useful sites for downstream hybridization or amplification reactions, as described below.

In embodiments, in which at least one phase of the log-linear amplification reaction is a PCR-based polymerization reaction, the absolute and relative concentrations of the exponential and linear primers can be used to establish appropriate conditions for carrying out a log-linear amplification. For example, as described above, the reaction conditions of a log-linear amplification are designed so that the exponential phase of the reaction terminates before reaching a plateau. In some embodiments, this can be accomplished by utilizing a limiting concentration of at least one of the exponential primers. When the “limiting primer” is consumed by the exponential phase, double-stranded amplicon synthesis terminates. Therefore, by “limiting concentration of an exponential primer” is meant a concentration of at least one exponential primer that results in the termination of the exponential phase prior to reaching a plateau. In some embodiments, the amount of primer can be adjusted so that a selected number of exponential amplicons are generated.

Determining the appropriate limiting concentration of one or more amplification primers is within the abilities of the skilled artisan. Non-limiting examples of factors to be considered include, the quantity of target sequence, the relative amount of each target polynucleotide sequence to be amplified, the number of different target polynucleotides sequences amplified in a single reaction (i.e., multiplex or single-plex), the sensitivity of the detection system, and the degree of accuracy desired. In various exemplary embodiments, the concentration of the limiting primer is less than about 50 nM, less than about 40 nM, less than about 30 nM, less than about 20 nM, or less than about 10 nM. In another embodiment, the concentration of the limiting primer is about 10 nM to about 30 nM.

Other methods may be used to terminate the exponential reaction prior to plateau and/or when a desired number of double-stranded amplicons are produced. In some embodiments, the exponential phase reaction can be monitored in real-time using, for example, fluorescent probe or dye chemistries as is known in the art (see, e.g., U.S. Pat. Nos. 5,210,015, 5,487,972, 5,804,375, 6,214,979 and WO 92/02638). When a detectable signal is produced as a result of double-stranded amplicon synthesis, the reaction can be terminated by one or more methods. For example, the exponential reaction can be terminated by the addition of one or more probes complementary to one or more of the exponential amplification primers, by the addition of an antibody (e.g., monoclonal or polyclonal antibody) or antigen binding fragments thereof (Fab, Fab′, F(ab′)₂, Fv (single chain antibody), chimeric antibodies, etc., either produced by the modification of whole antibodies or those synthesized de novo using recombinant DNA technologies that specifically binds to the polymerase. (see, e.g., Harlow et al. Antibodies: A Laboratory Manual (Cold Spring Harbor Laboratory Press, 1988)) or by the addition of a compound, such as a chelator (e.g., EDTA) to sequester a co-factor required for polymerase activity (e.g., Mg²⁺).

In the embodiments, in which, the concentration of only one exponential amplification primer is limiting, the non-limiting exponential primer can function as the linear primer after the limiting exponential primer is consumed. In such embodiments, the non-limiting exponential primer can be included in the log-linear amplification reaction in excess concentration. While the actual concentration is not critical for success, skilled artisans will appreciate that the primer should be included at concentrations high enough to support the desired degree of linear amplification without interfering with the overall log-linear amplification. In some embodiments, the non-limiting exponential primer can be included in a log-linear amplification at an initial concentration that is at least about 50-fold greater than the initial concentration of the limiting exponential primer. In other embodiments, the concentration of the non-limiting exponential primer is at least about 60-fold greater, at least about 70-fold greater, at least about 80-fold greater, at least about 90-fold greater, at least about 100-fold greater or at least about 500-fold greater.

In embodiments in which the concentrations of both exponential amplification primers are limiting, a third primer can be utilized as the linear primer. In these embodiments, the linear primer can be designed to hybridize to either strand of the double-stranded amplicons at a position that overlaps with an exponential primer or that is 3′ relative thereto. In some embodiments, the linear primer hybridizes to a sequence introduced into the double-stranded amplicons by an exponential amplification primer. As described above, amplification primers, including the exponential primers, may optionally include regions of mismatch or sequence diversity in comparison to the target sequence. Although these regions do not hybridize to the target sequence, these regions and their complement are incorporated into the double-stranded amplicons during the exponential phase. For purposes of exemplification and not limitation, if a forward exponential primer contains a 5′-code sequence, the code sequence is incorporated into the first strand of the double-stranded amplicons and a sequence complementary thereto is incorporated into the second strand. Therefore, in some embodiments, to prime linear amplification, the linear primer may contain a sequence suitable for hybridizing to the complement of the code sequence.

In contrast to the exponential primer(s), the concentration of the linear primer is generally non-limiting. By “non-limiting concentration of linear primer” herein is meant that the linear primer concentration is in excess of the double-stranded amplicons produced by the exponential phase, when the exponential phase terminates. Therefore, the linear primer concentration is not rate-limiting in connection with the linear phase reaction. The skilled artisan is aware that as the linear phase proceeds, linear primer concentration decreases and is eventually consumed. However, to accurately quantitate a target sequence by log-linear amplification, the linear primer concentration should be at least non-limiting at the point when the exponential phase terminates. Determining a suitable non-limiting concentration of linear primer is within the abilities of the skilled artisan and non-limiting examples of factors to be considered are described above. However, in various exemplary embodiments, the concentration of the linear primer can be at least about 100 nM, to at least about 500 nM, to at least about 1 μM or even greater. In relation to the number of exponential amplicons produced, the linear primer can be at least about 2 times higher than the exponential amplicons, to at least about 10 times higher, to at least about 20 times higher, or even higher.

In some embodiments, an amplification primer is a nucleobase polymer. By “nucleobase” is meant naturally occurring and synthetic heterocyclic moieties commonly known to those who utilize nucleic acid or polynucleotide technology or utilize polyamide or peptide nucleic acid technology to generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine). Other non-limiting examples of suitable nucleobases include those nucleobases disclosed in FIGS. 2(A) and 2(B) of Buchardt et al. (U.S. Pat. No. 6,357,163, WO 92/20702 and WO 92/20703).

Nucleobases can be linked to other moieties to form nucleosides, nucleotides, and nucleoside/tide analogs. As used herein, “nucleoside” refers to a compound consisting of a purine, deazapurine, or pyrimidine nucleoside base, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose. When the nucleoside base is purine or 7-deazapurine, the pentose is attached at the 9-position of the purine or deazapurine, and when the nucleoside base is pyrimidine, the pentose is attached at the 1-position of the pyrimidine (see, e.g., Kornberg and Baker, DNA Replication, 2nd Ed. (Freeman 1992)). The term “nucleotide” as used herein refers to a phosphate ester of a nucleoside, e.g., a mono-, a di-, or a triphosphate ester, wherein the most common site of esterification is the hydroxyl group attached to the C-5 position of the pentose. “Nucleotide 5′-triphosphate” refers to a nucleotide with a triphosphate ester group at the 5′ position. The term “nucleoside/tide” as used herein refers to a set of compounds including both nucleosides and/or nucleotides.

“Nucleobase polymer or oligomer” refers to two or more nucleobases connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, to several hundred nucleobases, to several thousand nucleobases, or more.

“Polynucleotide or oligonucleotide” refers to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

In some embodiments, a nucleobase polymer is an polynucleotide analog or an oligonucleotide analog. By “polynucleotide analog or oligonucleotide analog” is meant nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani, 1995, Chem. & Eng. News 4-5:1153; Dempey et al., 1995, J. Am. Chem. Soc. 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al., 2002, Biochemistry 41:9973-9981; Koshkin et al., 1998, J. Am. Chem. Soc. 120:13252-3; Koshkin et al., 1998, Tetrahedron Letters, 39:4381-4384; Jumar et al., 1998, Bioorganic & Medicinal Chemistry Letters 8:2219-2222; Singh and Wengel, 1998, Chem. Commun., 12:1247-1248; WO 00/56746; WO 02/28875; and, WO 01/48190.

In some embodiments, a nucleobase polymer is a polynucleotide mimic or oligonucleotide mimic. “Polynucleotide mimic or oligonucleotide mimic” refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. Nos. 5,786,461, 5,766,855, 5,719,262, 5,539,082 and WO 98/03542 (see also, Haaima et al., 1996, Angewandte Chemie Int'l Ed. in English 35:1939-1942; Lesnick et al., 1997, Nucleosid. Nucleotid. 16:1775-1779; D'Costa et al., 1999, Org. Lett. 1:1513-1516; Nielsen, 1999, Curr. Opin. Biotechnol. 10:71-75); uncharged polyamide backbones as described in WO 92/20702 and U.S. Pat. No. 5,539,082; uncharged morpholino-phosphoramidate backbones as described in U.S. Pat. No. 5,698,685, U.S. Pat. No. 5,470,974, U.S. Pat. No. 5,378,841 and U.S. Pat. No. 5,185,144 (see also, Wages et al., 1997, BioTechniques 23:1116-1121); peptide-based nucleic acid mimic backbones (see, e.g., U.S. Pat. No. 5,698,685); carbamate backbones (see, e.g., Stirchak and Summerton, 1987, J. Org. Chem. 52:4202); amide backbones (see, e.g., Lebreton, 1994, Synlett. February, 1994:137); methylhydroxylamine backbones (see, e.g., Vasseur et al., 1992, J. Am. Chem. Soc. 114:4006); 3′-thioformacetal backbones (see, e.g., Jones et al., 1993, J. Org. Chem. 58:2983) and sulfamate backbones (see, e.g., U.S. Pat. No. 5,470,967). All of the preceding references are herein incorporated by reference.

“Peptide nucleic acid” or “PNA” refers to poly- or oligonucleotide mimics in which the nucleobases are connected by amino linkages (uncharged polyamide backbone) such as described in any one or more of U.S. Pat. Nos. 5,539,082, 5,527,675, 5,623,049, 5,714,331, 5,718,262, 5,736,336, 5,773,571, 5,766,855, 5,786,461, 5,837,459, 5,891,625, 5,972,610, 5,986,053, 6,107,470, 6,451,968, 6,441,130, 6,414,112 and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorganic & Medicinal Chemistry Letters, 4:1081-1082; Petersen et al., 1996, Bioorganic & Medicinal Chemistry Letters, 6:793-796; Diderichsen et al., 1996, Tett. Lett. 37:475-478; Fujii et al., 1997, Bioorg. Med. Chem. Lett. 7:637-627; Jordan et al., 1997, Bioorg. Med. Chem. Lett. 7:687-690; Krotz et al., 1995, Tett. Lett. 36:6941-6944; Lagriffoul et al., 1994, Bioorg. Med. Chem. Lett. 4:1081-1082; Diederichsen, 1997, Bioorg. Med. Chem. 25 Letters, 7:1743-1746; Lowe et al., 1997, J. Chem. Soc. Perkin Trans. 1, 1:539-546; Lowe et al., 1997, J. Chem. Soc. Perkin Trans. 11:547-554; Lowe et al., 1997, I. Chem. Soc. Perkin Trans. 1 1:555-560; Howarth et al., 1997, I. Org. Chem. 62:5441-5450; Altmann et al., 1997, Bioorg. Med. Chem. Lett., 7:1119-1122; Diederichsen, 1998, Bioorg. Med. Chem. Lett., 8:165-168; Diederichsen et al., 1998, Angew. Chem. mt. Ed., 37:302-305; Cantin et al., 1997, Tett. Lett., 38:4211-4214; Ciapetti et al., 1997, Tetrahedron, 53:1167-1176; Lagriffoule et al., 1997, Chem. Eur. 1. '3:912-919; Kumar et al., 2001, Organic Letters 3 (9):1269-1272; and the Peptide-Based Nucleic Acid Mimics (PENAMs) of Shah et al. as disclosed in WO 96/04000.

Some examples of PNAs are those in which the nucleobases are attached to an N-(2-aminoethyl)-glycine backbone, i.e., a peptide-like, amide-linked unit (see, e.g., U.S. Pat. No. 5,719,262; Buchardt et al., 1992, WO 92/20702; Nielsen et al., 1991, Science 254:1497-1500).

In some embodiments, a nucleobase polymer is a chimeric oligonucleotide. By “chimeric oligonucleotide” is meant a nucleobase polymer or oligomer comprising a plurality of different polynucleotides, polynucleotide analogs and polynucleotide mimics. For example a chimeric oligo may comprise a sequence of DNA linked to a sequence of RNA. Other examples of chimeric oligonucleotides include a sequence of DNA linked to a sequence of PNA, and a sequence of RNA linked to a sequence of PNA.

As discussed above in connection with Equations (1) to (5), the copy number of various polynucleotides sequences present in a sample can be determined in a log-linear amplification reaction based upon the rate of accumulation of linear amplification product, and in some embodiments, the amount of linear amplicon present in the reaction at a specified point in time (e.g., after a specified cycle number). In some embodiments, the accumulation of linear amplicons and/or the rate of linear amplification can be monitored in real-time by carrying out the log-linear amplification reaction in the presence of a reporter molecule that generates a detectable signal proportion to the amount of linear amplicon present in the reaction. By “reporter molecule” herein is meant a molecule that produces a differential signal when specifically or non-specifically bound to a single-stranded polynucleotide relative to the unbound molecule. Non-limiting examples of reporter molecules include sequence-independent binding agents and sequence-specific binding agents. By “sequence-independent binding” is meant differential binding that is based on structure other than the sequence of a polynucleotide. Therefore, non-limiting examples of structure-specific binding agents include intercalating agents, such as, actinomycin D which fluoresces red when bound to single-stranded polynucleotides and green when bound to double-stranded polynucleotides. By “sequence-specific binding” is meant differential binding based on the sequence of a polynucleotide. Therefore, in some embodiments, a sequence-specific reporter molecule is an oligonucleotide probe. Such oligonucleotide probes include, but are not limited to, hydrolyzable probes (see, e.g., 5′-nuclease probes, (e.g., self-quenching fluorescent probes, e.g., TaqMan® probes), various stem-loop molecular beacons (see, e.g., U.S. Pat. Nos. 6,103,476 and 5,925,517 and Tyagi and Kramer, 1996, Nature Biotechnology 14:303-308), stemless or linear beacons (see, e.g., WO 99/21881), PNA molecular beacons (see, e.g., U.S. Pat. No. 6,355,421), linear PNA beacons (see, e.g., Kubista et al., 2001, SPIE 4264:53-58), non-FRET probes (see, e.g., U.S. Pat. No. 6,150,097) and the various different sunrise primers, scorpion probes, cyclicons (Kandimalla et al., 2000, Bioorg Med. Chem. 8 (8):1911-6), peptide nucleic acid (PNA) light-up probes, self-assembled nanoparticle probes (Taton et al., 2000, Science. 289 (5485): 1757-60), dual-probe systems, and ferrocene-modified probes described, for example, in U.S. Pat. No. 6,485,901; Mhlanga et al., 2001, Methods. 25:463-471; Whitcombe et al., 1999, Nat. Biotechnol. 17:804-807; Isacsson et al., 2000, Mol Cell Probes. 14:321-328; Svanvik et al., 2000, Anal. Biochem. 281:26-35; Wolffs et al., 2001, Biotechniques. 766:769-771; Tsourkas et al., 2002, Nucleic Acids Res. 30:4208-4215; Riccelli et al., 2002, Nucleic Acids Res. 30:4088-4093; Zhang et al., 2002, Shanghai. 34:329-332; Maxwell et al., 2002, J Am Chem Soc. 124:9606-9612; Broude et al., 2002, Trends Biotechnol. 20:249-56; Huang et al., 2002, Chem Res Toxicol. 15:118-126; and Yu et al., 2001, J. Am. Chem. Soc. 14:11155-11161) and hydrolyzable “flap” probes comprising a sequence suitable for hybridizing to a target polynucleotide (target polynucleotide specific sequence) and a cleavage or flap sequence that is not suitable for hybridizing to a target polynucleotide. Therefore, when the “flap” probe is hybridized to a target polynucleotide the cleavage sequence forms a single-stranded region, which may be released from the probe by the 5′-3′ nuclease activity of a polymerase. The released cleavage sequence may be detected by various methods, including but not limited to, capillary electrophoresis and other methods as described in U.S. Patent Application Serial Nos. U.S. Patent Application Ser. Nos. 60/584,621, 60/584,596, 60/584,643, each filed Jun. 30, 2004.

Similar to the primers discussed above, the oligonucleotide probes may be nucleobase polymers, such as, but not limited to, DNA, RNA, PNA, or chimeras composed of one or more combinations thereof. The oligonucleotides probes may be composed of standard or non-standard nucleobases or mixtures thereof and may include one or more modified interlinkages, as previously described in connection with the amplification primers. The oligonucleotide probes are suitable to produce a detectable signal proportional to the number of single-stranded amplicons produced by the linear phase. Therefore, in some embodiments, a probe has a moiety or label suitable for producing a detectable signal. Exemplary labels include but are not limited to fluorophores and chemiluminescent labels. Such labels allow direct detection of labeled compounds by a suitable detector, e.g., a fluorometer. In some embodiments, the label is a fluorogenic moiety detectable by a fluorometer and forms part of a signal-quencher dye pair. In other embodiments, the label is a fluorogenic reporter dye detectable by a fluorometer and forms part of a signal-donor dye pair. In multiplex log-linear embodiments, in which a plurality of target polynucleotides are analyzed in a single long-linear amplification reaction, a plurality of probes specific for each target polynucleotide can be employed, in which, each probe has a distinguishable label such that the different linear amplicons can be detected or monitored in a single reaction vessel.

In some embodiments, an oligonucleotide probe is substantially complementary to at least a region of a single-stranded amplicon. In other embodiments, an oligonucleotide probe is substantially complementary to at least a region of one strand of a double-stranded amplicon at a position 3′ relative to the linear primer, e.g., a 5′-nuclease probe, “flap” probe). In some embodiments, the position 3′ relative to the linear primer is the target sequence. In other embodiments, the position 3′ relative to the linear primer comprises a code sequence or a sequence complementary thereto. By “substantially complementary” herein is meant that the sequences of the probe include enough complementarity to hybridize to either the single-stranded amplicon or to one strand of a double-stranded amplicon 3′ relative to the linear primer and produce a detectable signal directly proportional to the number of single-stranded amplicons. Therefore, in some embodiments, oligonucleotide probes may be completely complementary or contain regions of mismatch or non-complementarity as described above. The exact degree of complementarity will depend upon the desired application for the probe and will be apparent to those of skill in the art. However, for purposes of quantitating a target sequence, the hybridization of the probe is suitable to produce a detectable signal at a rate proportional to the number of double-stranded amplicons, and the magnitude of the detectable signal at any time point or cycle number during the linear phase is proportional to the number of single-stranded amplicons.

The lengths of such oligonucleotide probes can vary broadly, and in some embodiments can range from as few as two to as many as tens or hundreds of nucleotides, depending upon the particular application for which the probe is designed. In one embodiment, the oligonucleotide probes range in length from about 15 to 35 nucleotides. In another embodiment, the oligonucleotide probes range in length from about 15 to 25 nucleotides. The exact lengths of the probes may depend on many factors, such the factors described above in connection with the design of primers. Therefore, a skilled artisan will appreciate, the general principles and methods applied to the design of primers also apply to the design of probes. To ensure the binding of a probe to each linear amplicon that is produced, a molar excess of probe relative to linear primer may be used. In various exemplary embodiments, the concentration of the probe is at least about 2 times higher than the linear primer concentration, to at least about 10 times higher, to at least about 20 times higher, or even higher.

As amplification proceeds, the reporter molecule produces a detectable signal. Neither the reporter molecule nor the detectable signal prevents or substantially interferes with log-linear amplification from proceeding. For practicing the disclosed methods, a reporter molecule is suitable for log-linear amplification, so long as in the presence of the reporter molecule a net increase in the amount of single-stranded amplicons present is reflected in a change in signal intensity that is detectable directly or indirectly.

In alternative embodiments, the accumulation of linear amplicons can be assessed without the aide of a reporter molecule. For example, linear amplicons produced in a single-plex or multiplex format may be detected at one or more time points by capillary electrophoresis. In a multiplex format, the linear amplicons can be advantageously designed to be of different lengths to facilitate their electrophoretic separation and identification. Alternatively, the linear amplicons can be designed to include mobility modifiers as is well-known in the art (see, e.g., U.S. Pat. Nos. 5,470,705, 5,514,543, 6,395,486, and 6,734,296).

In some embodiments, the linear primer may optionally contain a moiety that is capable of producing a detectable signal. For example, in these embodiments, the linear primer is distinct from the two exponential primers, i.e., a three-primer, rather than a two-primer, log-linear reaction is utilized to avoid incorporation of the detectable moiety into the exponential amplicons. Therefore, each linear amplicon contains a detectable moiety. In these embodiments, various downstream assays may be used to detect and quantify the linear amplicons. In one embodiment, the linear amplicons can be quantitated by capillary electrophoresis. For example, once a linear primer containing a detectable a moiety is extended by the action of a polymerase to produce a linear amplicon, the linear amplicon and unextended primers are electrophoretically separated and the linear primers can be identified and quantitated. For multiplex embodiments, linear amplicons can be designed to be different in length to facilitate their electrophoretic separation and individual quantitation.

In some embodiments, the detectable signal is measured at one or more discrete time points or is continuously monitored in real-time. In these embodiments, continuous or discrete monitoring may utilize a reporter molecule comprising a fluorophore-quencher pair. Detection of the fluorescent signal can be performed in any appropriate way based, in part, upon the type of reporter molecule employed (e.g., 5′-nuclease probe vs. a molecular beacon) as known in the art. In some embodiments, the signal is compared against a control signal (e.g., before start of the modification), threshold signal, or standard curve.

As discussed above, depending upon the nature of the sample polynucleotides to be amplified (e.g., RNA or DNA), a log-linear amplification can be accomplished by using well-known principles and reagents for PCR or RT-PCR. Thus, log-linear amplifications in which the target polynucleotide(s) is a DNA will typically include as essential components, in addition to the amplification primers, a mixture of 2′-deoxribonucleoside triphosphates (dNTPs) suitable for template-dependent DNA synthesis (e.g., primer extension) and a DNA polymerase. Multiplex/single-plex amplifications in which the target polynucleotide(s) is RNA will typically additionally include a reverse-transcriptase. In some embodiments, one or more of the amplification primers are suitable for priming reverse transcription. In other embodiments, a primer specifically designed to prime reverse transcription is used. With the exception of certain parameters described herein, log-linear amplification reactions may be carried out using reagents, reagent concentrations and reaction conditions conventionally employed in such conventional PCR and RT-PCR reactions. For example, except as noted herein, enzymes (e.g. DNA polymerases and reverse transcriptases), enzyme concentrations, dNTP mixtures (as well as their absolute and/or relative concentrations), total target polynucleotide concentrations, buffers, buffer concentrations, pH ranges, cycling times and cycling temperatures employed in conventional PCR and RT-PCR reactions may be used for the log-linear amplification reactions. Guidance for selecting suitable reaction conditions may be found, for example, in U.S. Pat. Nos. 4,683,202, 4,683,195, 4,800,159, 4,965,188, 5,561,058, 5,618,703, 5,693,517, 5,876,978, 6,087,098, 6,436,677 and 6,485,917, and PCR Essential Data, (C. R. Newton ed. 1995) and PCR Protocols: A Guide to Methods and Applications. (Innis, M, Gelfand, D., Sninsky, J. and White, T., eds. 1990). A variety of tools for designing PCR and RT-PCR amplification primers, as well as myriad protocols, reaction conditions and techniques for carrying out various different types of PCR reactions, including conventional PCR reactions and RT-PCR reactions are also well known (see, e.g., U.S. Pat. Nos. 4,683,195; 4,988,617, 5,891,625; Bustin (ed). A-Z of Quantitative PCR (IUL Biotechnology, No. 5) (IUL Biotechnology Series) International University Line (July 2004) (ISBN: 0963681788); Edwards et al. Real-Time PCR: An Essential Guide (Horizonbioscience) (BIOS Scientific Publishers (Jul. 1, 2004) (ISBN: 095452327X); Innis et al. PCR Protocols: A Guide to Methods and Applications (ISBN: 0123721814); Weissensteiner et al. (eds). PCR Technology: Current Innovations, CRC Press; 2nd edition (Nov. 1, 2003) (ISBN: 0849311845).). All of these various tools and protocols can be used in connection with the log-linear amplification reactions described herein.

Like conventional PCR and RT-PCR amplification reactions, the log-linear amplification reactions may be carried out with a variety of different DNA polymerases. In some embodiments, the DNA polymerase also has 5′-3′ endonuclease activity. In some embodiments, the DNA polymerase is a thermostable polymerase. In some embodiments, the DNA polymerase polymerase has 5′-3′ nuclease activity. Non-limiting examples of polymerases with 5′-3′ nuclease activity include, but are not limited to, AmpliTaq® DNA polymerase, Ampli-Taq® GOLD polymerase and Tth polymerases (Applied Biosystems, Foster City, Calif.), E. coli DNA polymerase I (New England Biolabs, Beverly, Mass.), rBst DNA Polymerase (Epicenter®, Madison, Wis.), and Tfl DNA polymerase (Promega Corp., Madison, Wis.). Moreover, like conventional RT-PCR amplification reactions, log-linear RT-PCR amplification reactions may be carried out with a variety of different reverse transcriptases, although in some embodiments thermostable reverse-transcriptases are preferred. Suitable thermostable reverse transcriptases include, but are not limited to, reverse transcriptases such as AMV reverse transcriptase, MuLV, and Tth reverse transcriptase. Temperatures suitable for carrying out the various denaturation, annealing and primer extension reactions with the polymerases and reverse transcriptases are well-known in the art. Optional reagents commonly employed in conventional PCR and RT-PCR amplification reactions, such as reagents designed to enhance PCR, modify Tₘ, or reduce primer-dimer formation, may also be employed in the log-linear amplification reactions. (see, e.g., U.S. Pat. Nos. 6,410,231; 6,482,588; 6,485,903; and 6,485,944, all of which are incorporated herein by reference.) In certain embodiments, the log-linear amplifications may be carried out with commercially-available amplification reagents, such as, for example, AmpliTa® Gold PCR Master Mix, TaqMa® Universal Master Mix and TaqMan® Universal Master Mix No AmpErase® UNG, all of which are available commercially from Applied Biosystems (Foster City, Calif.).

While the exponential phase of the log-linear amplification described herein has been exemplified and described with reference to PCR and/or RT-PCR applications, skilled artisans will recognize that other types of exponential amplification reactions may be utilized. For example, in some embodiments, the exponential phase of the reaction is carried out using well-known principles and reagents of the ligase chain reaction (LCR), in which double-stranded ligation amplicons are produced by multiple rounds of thermocycling in the presence of a thermostable ligase (see, e.g, EP-A-320308 and U.S. Pat. Nos. 5,427,930, 5,516,663, 5,686,272 and 5,869,252).

Exponential amplification by LCR utilizes four ligation probes. In one example of LCR, first and second ligation probes hybridize to a single-stranded target sequence to form a substrate for a ligase, which ligates the first and second ligation probes. Third and fourth ligation probes hybridize to the ligated first and second probes and are ligated, thereby producing a double-stranded ligation amplicon. In another example, wherein the target polynucleotide is double-stranded, the third and fourth ligation probes also hybridize to the complement of the target sequence and are ligated. However, in both examples, by repeated cycles of denaturation, hybridization, and ligation, the number of copies of the double-stranded ligation amplicons exponentially increases by a theoretical factor of two. Therefore, the methods described above for conducting log-linear amplification using PCR principles are applicable to a log-linear reaction in which the target sequence is exponentially amplified by LCR.

In some embodiments, the ligation probes may contain regions of mismatch as described above, that are incorporated into the double-stranded ligation amplicons to provide useful cites for downstream hybridization or amplification reactions, such as the linear phase reaction. Thus, the double-stranded ligation amplicons may be linearly amplified as described above. Alternatively, linear amplification may proceed using a linear ligation reaction or the isothermal linear amplification, as described below.

Skilled artisans will recognize that other types of linear amplification reactions also may be utilized in any combination with the above-describe exponential phase amplification reactions. For example, in some embodiments, an isothermal linear phase amplification reaction may be coupled to virtually any exponential phase reaction. In the isothermal linear phase, the double-stranded amplicons produced by the exponential phase may be transcribed to produce single-stranded RNA amplicons. Transcription occurs in the presence of a DNA-dependent RNA polymerase (RNA polymerase, e.g., T4 polymerase or T7 polymerase) and rNTPs suitable for single-stranded RNA synthesis. The linear phase transcription does not require thermal cycling and, therefore, the linear phase is an isothermal reaction, incubated within a temperature that is optimum for the transcription enzyme and the degree of transcription desired.

The skilled artisan appreciates that transcription requires a promoter upstream from the sequence to be transcribed. Therefore, in some embodiments, a promoter sequence may be introduced into the double-stranded amplicons upstream from each target sequence by incorporating the promoter sequence into either the forward or reverse amplification primer. Therefore, the exponential phase produces double-stranded amplicons having an RNA promoter at or near the 5′ terminus of the first or second strand. Single-stranded RNA amplicon synthesis may be detected utilizing one or more of the reporter molecules, described above. However, reporter molecules that hybridize to the double-stranded amplicon are preferably avoided as these types of reporter molecules may interfere with transcription.

In some embodiments, the linear phase of the amplification reaction is a linear ligase amplification reaction. In some embodiments, the exponential amplicons contain adjacent regions of sequence diversity relative to the target sequence. For example, the first strand of the exponential amplicons may contain a 5′ universal sequence that is shared by all exponential amplicons produced from each target sequence. 3′ relative to the universal sequence is a code sequence, which as described above, is substantially unique for each exponential amplicon. The 3′ terminus of the second strand of the exponential amplicons contains sequences complementary to the first strand. Therefore, a first ligation probe comprising the universal sequence hybridizes to the 3′ terminus of the second strand and a second ligation probe comprising a code sequence hybridizes to the second strand adjacent to the first probe. In this example, the first probe contains a fluorescent moiety and the second probe contains a mobility modifier suitable for distinguishing the second “code” probe. When hybridized to the second strand of the double-stranded amplicon, the first and second ligation probes form a substrate that is ligated by the action of a ligase, such as, a thermostable ligase, to form a linear ligation amplicon. Each ligation amplicon contains the same universal sequence and fluorescent moiety at the 5′ terminus; however each ligation amplicon will contain a unique code sequence and mobility modifier. Therefore, in this example of this embodiment, the ligation amplicons are distinguished and quantitated by capillary electrophoresis.

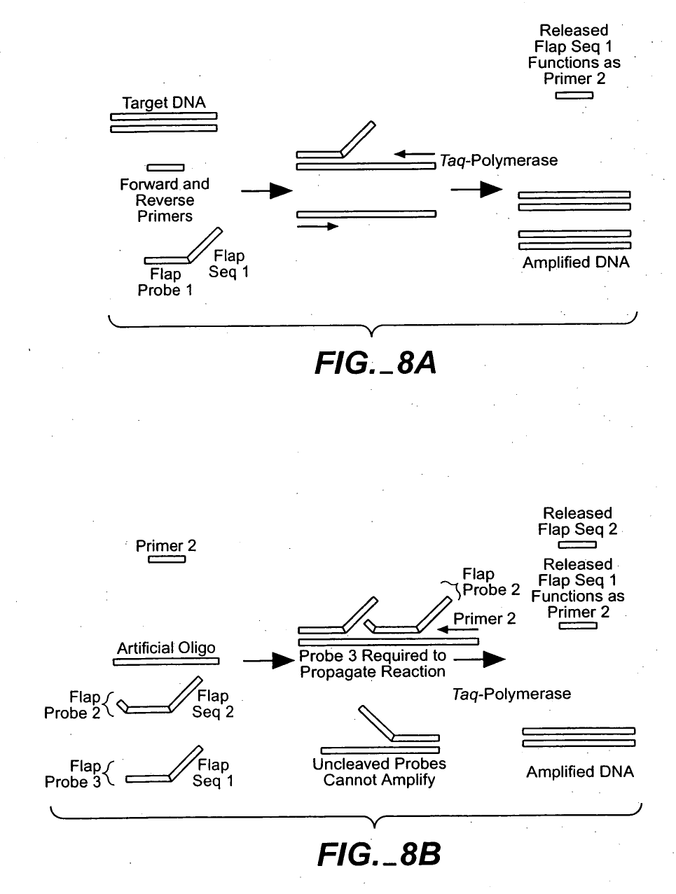

In the above described embodiments of log-linear amplification, the phases are coupled by the exponential amplicons functioning as templates for the linear phase amplification, in other embodiments, the phases can be coupled by a product of the exponential phase serving as a primer for the linear phase. For example, by exponentially amplifying a target sequence in the presence of a “flap” probe which hybridizes ₃′ relative to one of the exponential amplification primers, the “flap” is released by the nuclease activity of the polymerase. In some embodiments, the released “flap” sequence can be designed to function as a linear primer (FIG. 8A).

Furthermore, the coupled linear reaction can be achieved using several different approaches, including but not limited to, antibody or enzymatic reaction other than those described above. For example, in some embodiments the linear phase amplification can be a reaction in which each cycle of linear amplification generates more linear primer for subsequent rounds of linear amplicon synthesis. One non-limiting example of this type of linear phase amplification is illustrated in FIG. 8B. In this example, the linear phase amplification is carried out in the presence of a “flap” probe that hybridizes 3′ relative to a linear primer. In this example, the sequence of the “flap” is identical to the linear primer sequence. Therefore, release of the “flap” sequence by the nuclease activity of the polymerase generates additional linear primer.

The above described, log-linear amplification methods have general application to various types of nucleic acid based assays and techniques. For example, in addition to the detection and quantitation methods described above, in some embodiments, the linear amplicons may be detected and/or quantitated using array based assays (see, e.g., U.S. Pat. Nos. 5,412,087, 5,412,087, 5,489,678, 5,510,270, 5,549,974, 5,547,839, 5,482,867, 6,221,583, 6,232,062, 6,355,431, 6,396,995, 6,429,027, 6,544,732 and 6,620,584) or by single-strand conformational polymorphism analysis (see, e.g., Cha et al., 1997, Anal. Biochem. 252:24-32). In another non-limiting example, log-linear amplification may be used to produce templates for nucleic acid sequencing or for use in mutagenesis.

In another non-limiting example, the log-linear amplification may be used to determine the relative amounts of various target sequences in single-plex or multiplex formats. For example, gene expression analysis may be performed using, for example, the PCR-based log-linear amplification phases as described in which the amplification reaction is monitored in real-time or a single-time point measurement is made. Furthermore, such analyses may be made by directly measuring linear amplicon accumulation and/or by using a reporter molecule that serves as a proxy for the amount of linear amplicon. In another non-limiting example, gene expression analysis may be performed by coupling a linear ligase amplification to a PCR-based exponential reaction. As illustrated in FIG. 8, the exponential amplicons from samples and controls serve as templates for a linear ligase amplification reaction to produce ligation amplicons. In the control reaction, the first probe has an added nucleotide at its 5′ terminus. This provides a mobility shift such that the ligation amplicons from the sample amplification and the control amplification may be run on the same CE column for detection and quantitation. In addition,

The above-described log-linear amplification reaction provides advantages over conventional amplification techniques. For example, in some embodiments, log-linear amplification increases throughput of quantitative nucleic acid assays, such as, a conventional PCR, by decreasing the time required for measurements. In some embodiments, rather than monitoring log-linear amplification in real-time, a single point measurement may be utilized to determine target sequence copy number. In some embodiments, log-linear amplification provides an improved method of performing multiplex amplifications, such as, gene expression analysis. For example, in a conventional PCR, amplification primers are utilized at non-limiting concentrations. Therefore, if the dynamic range of the target sequences is very high, e.g., ˜10⁸, the highly expressed genes successfully compete with the lower expressed genes for PCR reagents. As a result, the highly expressed genes are over represented in the PCR amplicons. In addition, the lower expressed genes are under represented or may not be amplified to a detectable level. In some embodiments, log-linear amplification avoids this by using a limiting concentration of at least one exponential primer concentration to produce an equivalent number of double-stranded amplicons for each target sequence which are linearly amplified. Therefore, in some embodiments, rather than the amount of exponential amplicon produced for each target sequence, the time point or cycle number at which the exponential amplification phase terminates may be the indicator of the relative amount of each target sequence. Over representation of highly expressed genes are further minimized because the exponential phase amplification is converted to a linear phase during which linear amplicon production is measured either directly or indirectly using a one or more reporter molecules, as described above. Therefore, in these embodiments, the above-described limitations of a conventional PCR are avoided and increased sensitivity and accuracy are provided.

Also provided are kits for use in practicing the various embodiments of log-linear amplification. Therefore, in some embodiments, kits include one or more sets of exponential amplification primer pairs to log-linear amplify one or more target polynucleotide sequences. One or more of the exponential primers may be target sequence specific or may hybridize to sequences that flank the target sequence; however, the exponential primers are sufficiently long to prime log-linear amplification under the conditions of the reaction. Therefore, in some embodiments, the exponential primers contain from about 15 to about 35 nucleotides that are substantially complementary to the target polynucleotide, although in other embodiments these sequences may contain more of fewer nucleotides. In optional embodiments, one or more exponential amplification primers contains one or more sequences of diversity from the target polynucleotide. The sequences of diversity may be unique to one primer, e.g., a code sequence, or may be shared by two or more exponential primers. The sequences of diversity are incorporated into the double-stranded or single-stranded amplicons and provide convenient sites for downstream detection or analysis. One exponential primer of each pair is provided at a limiting concentration. The limiting exponential primer concentration is suitable to terminate exponential phase amplification at or about the cycle number that the selected number of double-stranded amplicons are produced. At this cycle number, the non-limiting exponential primer linearly amplifies the double-stranded amplicons to produce single-stranded amplicons.

In some embodiments, kits comprise one or more sets of exponential amplification primers and one or more linear primers. In this embodiment, the concentration of both exponential amplification primers are limiting and the concentration of the linear amplification primer(s) is non-limiting. The exponential primers are consumed at or about the cycle number that the selected number of double-stranded amplicons are produced. At that point, the linear primer continues to linearly amplify one strand of the double-stranded amplicons to produce single-stranded amplicons. In some embodiments, a kit includes one or more reporter molecules that produce a detectable signal proportional to the number of single-stranded amplicons. In other embodiments, a kit includes linear primer comprising a detectable moiety. In some embodiments, kits may comprise one or more sets of ligation probes for exponential and/or linear ligation, and/or one or more polymerases and/or ligases. Thus, in various embodiments, kits comprise compositions for carrying out log-linear amplification of at least one target sequence.

In some embodiments, the disclosed methods may be implemented on a general purpose or special purpose device, such as a device having a processor for executing computer program code instructions and a memory coupled to a processor for storing data and/or commands. It will be appreciated that the computing device may be a single computer or a plurality of networked computers and that the several procedures associated with implementing the methods and procedures described herein may be implemented on one or a plurality of computing devices. In some embodiments the disclosed procedures and methods are implemented on standard server-client network infrastructures with the inventive features added on top of such infrastructure or compatible therewith. Methods and procedures described herein generally may be implemented in software, hardware, or combinations thereof. Thus, in some embodiments, a device or apparatus comprises a thermal cycling system having a reaction module 20 linked to thermal control module 10. Reaction module 20 is optically linked 30 to an excitation source 50 and detector 60. The type of excitation source and detection system are selected at the discretion of the practitioner and are based on the detection method, and number and type of signals produced by the reporter molecule(s) employed. The apparatuses are further adapted to be operably linked to computer 70 that is directed by readable memory 80. The output of the computer is directed to an output device 90. The skilled artisan will appreciate, that the various components of the apparatus may have other configurations. In some embodiments, excitation source 50 and detector 60 may be may be in separate housings rather than contained in optical head 40. In some embodiments, processor 70 and output 90 device can be in the same housing. Non-limiting examples of existing apparatuses that may be used to carry out log-linear amplification and monitor the reaction in real-time or take one or more single time point measurements include, Models 7300, 7500, and 7700 Real-Time PCR Systems (Applied Biosystems, Foster City, Calif.); the MyCyler and iCycler Thermal Cyclers (Bio-Rad, Hercules, Calif.); the Mx3000P™ and Mx4000® (Stratagene®, La Jolla, Calif.); the Chromo 4™ Four-Color Real-Time System (MJ Research, Inc., Reno, Nev.); and the LightCycler® 2.0 Instrument (Roche Applied Science, Indianapolis, Ind.). In some embodiments, the computer readable memory 80 of such instruments is programmed with executable instructions to direct the computer to calculate the cycle number, Ce, at which a selected number of double-stranded amplicons are produced in the exponential phase of a log-linear reaction. In some embodiments, the computer readable memory directs the computer to determine when the selected number of exponential amplicons are produced, for example, when the exponential phase is monitored in real-time, and to terminate the exponential phase. In other embodiments, the computer readable memory is programmed to receive the intensity assigned to the measured signal, e.g., Iₘ, produced during the linear phase reaction and L, the rate of linear amplification. In other embodiments, the computer readable memory is programmed to receive measurements made during real-time monitoring of the linear phase and to determine therefrom an optimal Iₘ and L. In an optional embodiment, L is provided by the user from pre-determined standards or determined from a set of established standards run in parallel. In each of the above embodiments, the computer readable memory then directs the computer to calculate the target sequence copy number according to Equation (5).

The following examples are offered by way of illustration and not by way of limitation. All literature and similar materials cited in this application, including but not limited to, patents, patent applications, articles, books, and treatises, regardless of the format of such literature and similar materials, are expressly incorporated by reference in their entirety for any purpose. In the event that one or more of the incorporated literature and similar materials differs from or contradicts this application, including but not limited to defined terms, term usage, described techniques, or the like, this application controls.

### 6. EXAMPLES

### Example 1

### Log-Linear Amplification of 1 ag to 100 pg ATP5b Liver cDNA

Log-linear amplification was performed using various amounts of ATP5b cDNA. cDNA was quantitated and diluted from 100 pg (10⁷ copies), 10 pg (10⁶ copies), 1 pg (10⁵ copies; 1,000 fg), 100 fg (10⁴ copies), 10 fg (103 copies), 1 fg (10² copies; 1,000 ag), 100 ag (10⁻¹ copies), 10 ag (1 copy), and 1 ag (10⁻¹ copies) and log-linear amplified. ATP5b cDNA was exponentially amplified using ATP5b-UF (SEQ ID NO:5) and ATP5b-RP (SEQ ID NO:24). Universal forward primer (UF; SEQ ID NO: 14) was used as the linear primer. The reporter molecule was ATP5b-FAM⁺ (SEQ ID NO:25).

Log-linear master reaction mixture contained 12.5 μl 2×AB PCR Master Mix (Applied Biosystems, Foster City, Calif.), 2.5 μl 10 μM UF primer, 2.5 μl 200 nM ATP5b-UF primer, 2.5 μl 200 nM ATP5b-reverse primer (RP), 2.5 μl 5 μM ATP5B-FAM⁺, and 2.5 μl ddH₂O. To prepare final reaction mixtures 22.5 μl of the Master Max was added to 2.5 μl cDNA diluted as described above. Each reaction also contained the fluorophore ROX as an internal standard. The reactions were incubated at 50° C. for 2 min., 95° C. for 10 min. and thermocycled 80 times (95° C. for 15 sec., 60° C. for 1 min.). Fluorescent signals (FAM and ROX) were measured while the reaction was incubated at each 60° C./1 min-step and Iₘ was determined. Baseline fluorescence was measured during cycles 3-14 and ΔIₘ was determined.

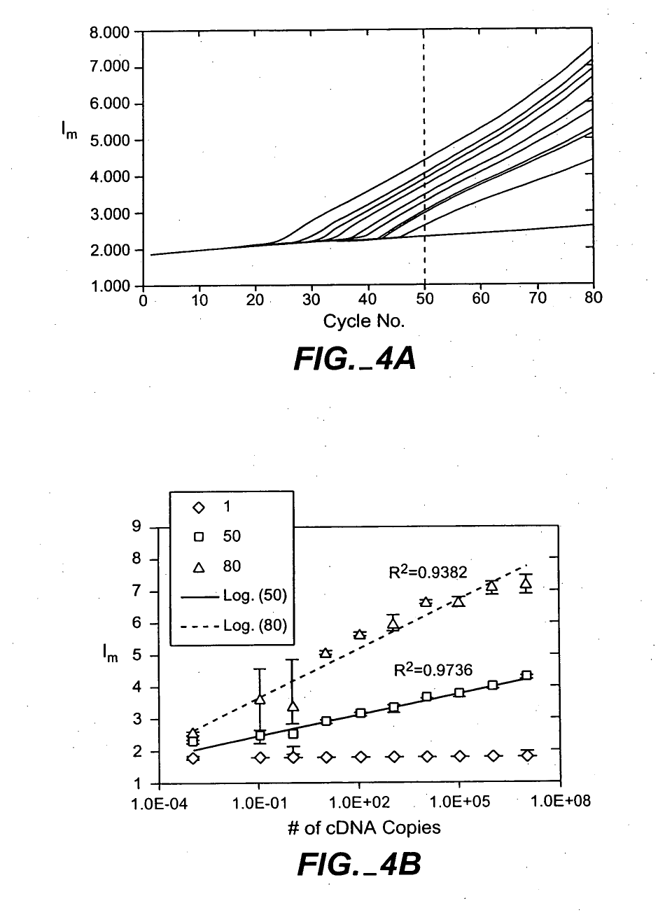

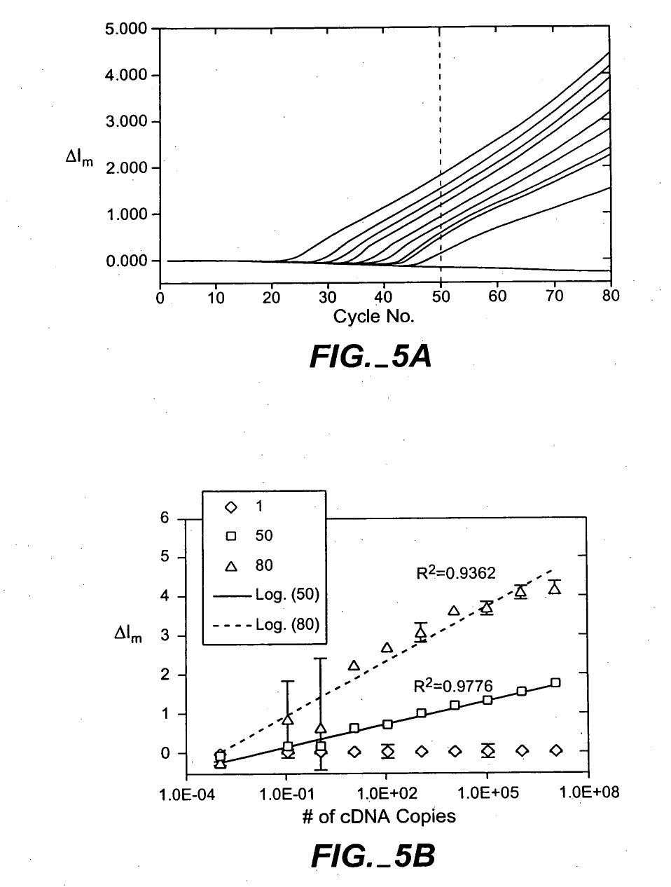

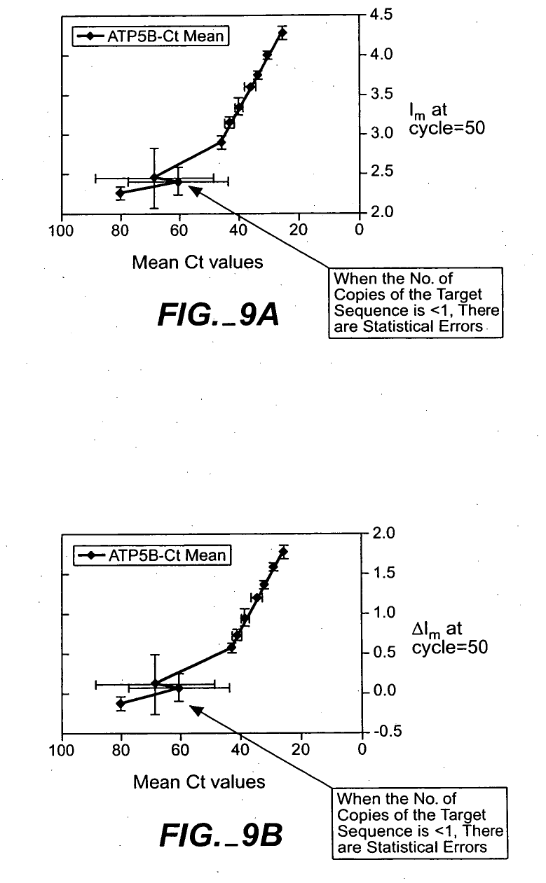

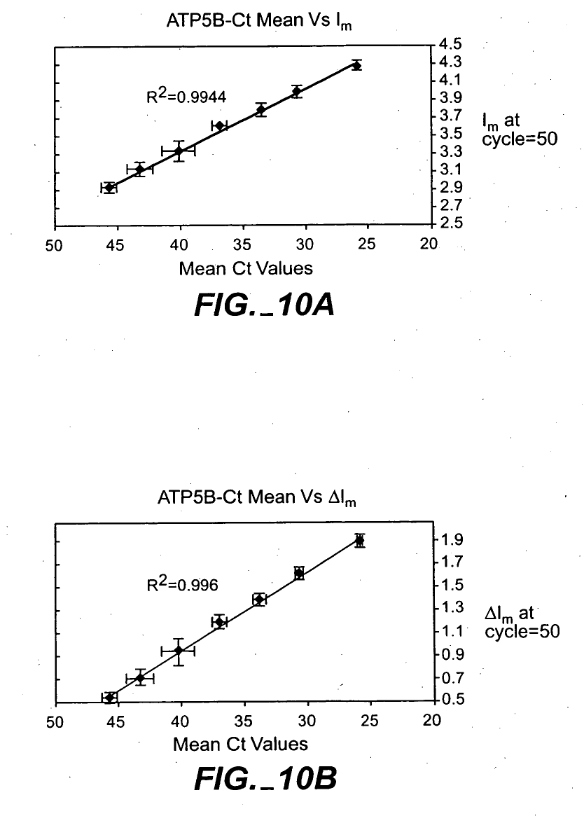

FIG. 4A is a graph of Iₘ vs. cycle number. FIG. 5A is a graph of ΔIₘ vs. cycle number. Each log-linear amplification reaction was also run in triplicate and a linear regression analysis for results obtained at cycle numbers 1, 50 and 80 are shown in FIGS. 4B and 5B. In each graph, R² at 50 cycles was closest to 1.0. Iₘ and ΔIₘ vs. mean Cₜ values are graphed in FIGS. 9A and 9B, respectively. The results indicate that statistical errors occurred when the target sequence copy numbers is less than 1. FIGS. 10A and 10B is a graph of the linear regression analysis of the data shown in FIGS. 9A and 9B at cycle number 50. For both graphs, R² obtained only varied from 0.9944 to 0.996.

### Example 2

### Log-Linear Amplification of 31 fg to 16 pg ATP5b Liver cDNA

Log-linear amplification was performed using various amount of ATP5b cDNA. The cDNA was quantitated and diluted from 16 pg (1.6×10⁶ copies), 8 pg (8×10⁵ copies), 4 pg (4×10⁵ copies), 2 pg (2×10⁵ copies), 1 pg (1×10⁵ copies), 0.5 pg (5×10⁴ copies; 500 fg), 250 fg (2.5×10⁴ copies), 125 fg (1.25×10⁴ copies), 62.5 fg (6.25×10³ copies), and 31 fg (3.1×10³ copies) and log-linear amplified. ATP5b cDNA was exponentially amplified using ATP5b-UF (SEQ ID NO:5) and ATP5b-RP (SEQ ID NO:22). Universal forward primer (UF; SEQ ID NO: 14) was used for linear amplification. The reporter molecule was ATP5b-FAM⁺ (SEQ ID NO:22).

The log-linear master reaction mixture, final reaction mixtures and the incubation and cycling conditions were same was described in Example 1.

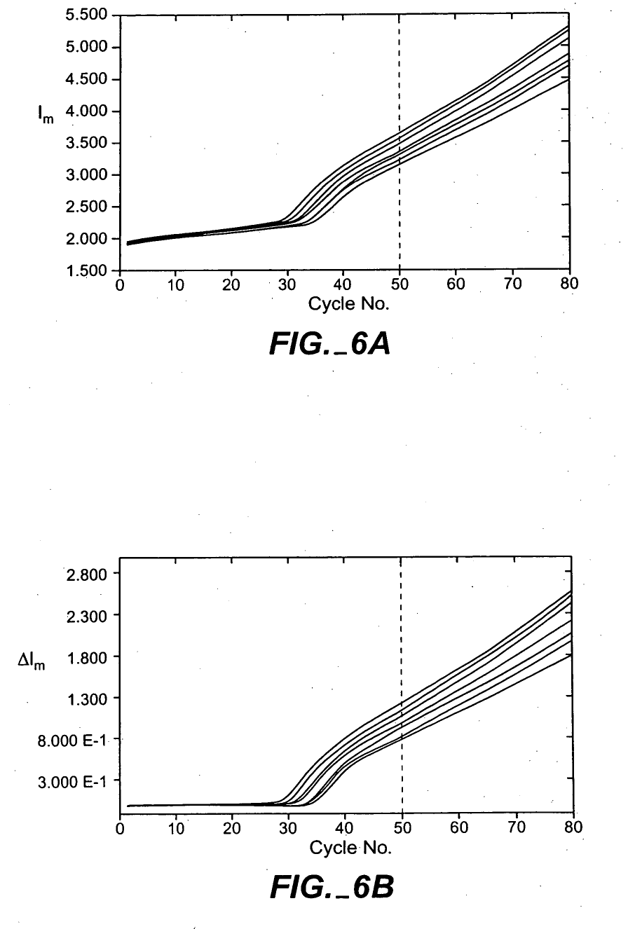

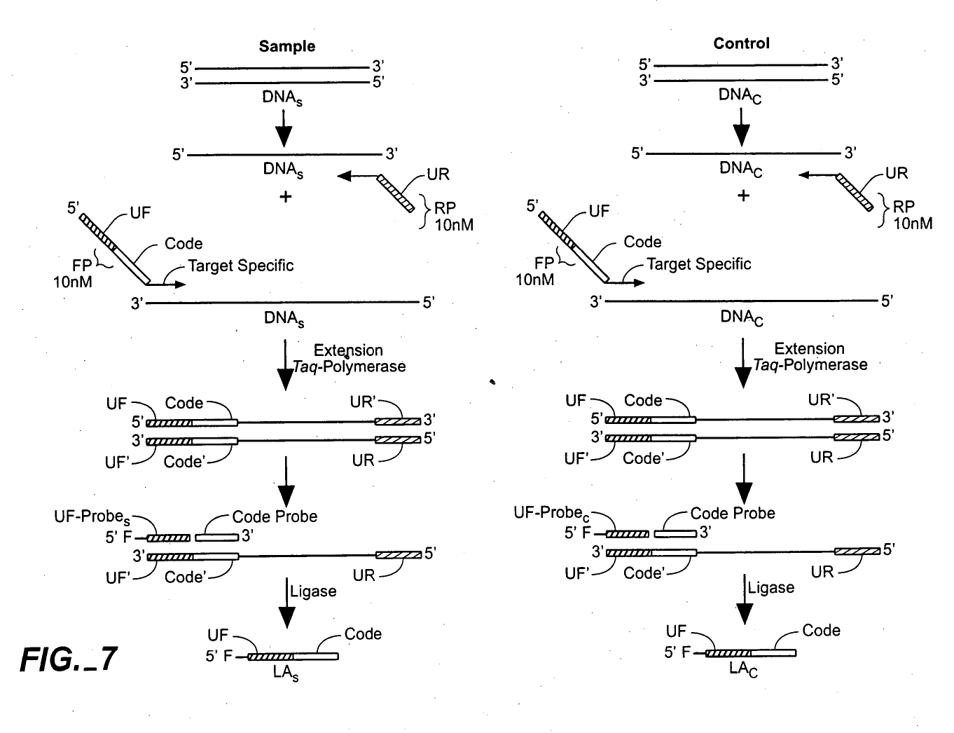

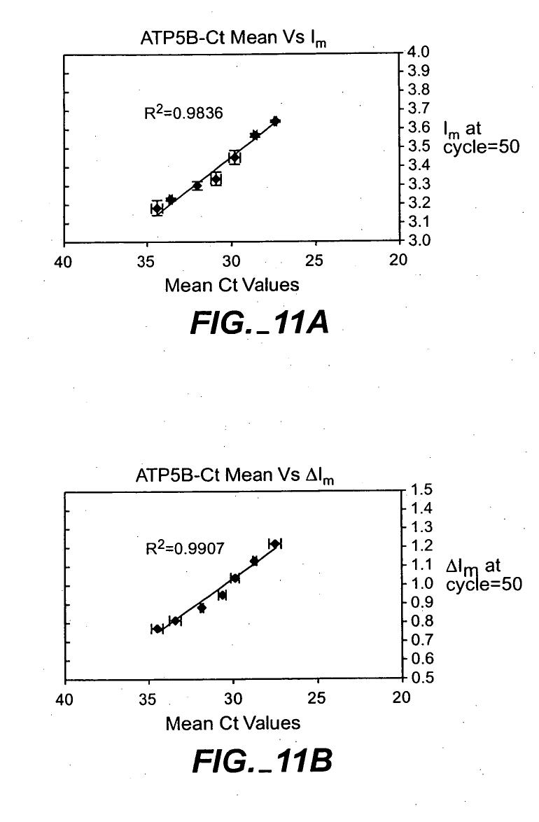

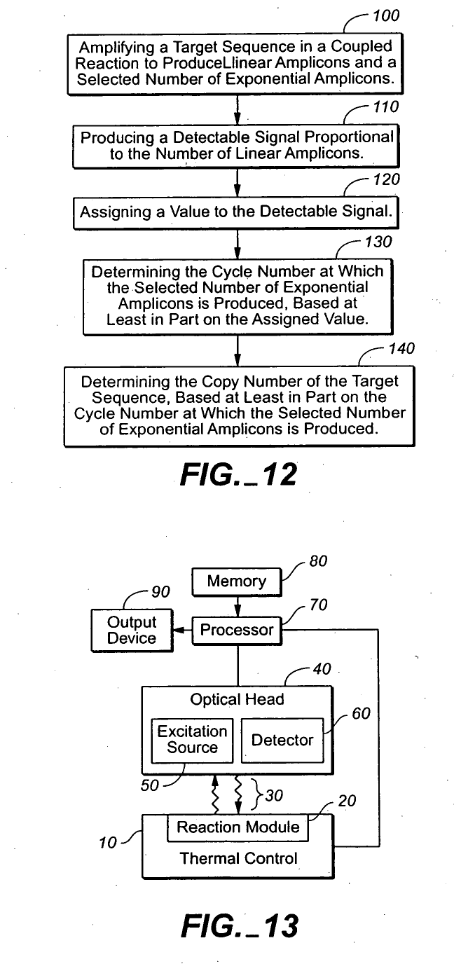

FIG. 6A is a graph of Iₘ vs. cycle number. FIG. 6B is a graph of ΔIₘ vs. cycle number. Each log-linear amplification reaction was also run in triplicate. A linear regression analysis for results obtained at cycle number 50 of Iₘ and ΔIₘ vs. mean Cₜ are shown in FIGS. 11A and 11B, respectively. In each graph, R² at 50 cycles only varied from 0.9836 to 0.9907.

TABLE 1

Sequence

| | | |
|---|---|---|
| Nucleic Acid | Sequence | Identifier |

| | | |
|---|---|---|
| ATP5B-FAM | FAM-5′-ATGGCTGAGACAAGAA-MGB | SEQ ID NO:1 |
| CETP-Vic | Vic-5′-AGCTGCTCTCAGTCAA-MGB | SEQ ID NO:2 |
| EIF1A-Ned | Ned-5′-CACCCGGCCCACGG-MGB | SEQ ID NO:3 |
| Cox6b-ROX | ROX-5′-ACACGCTGGTACCATT-MGB | SEQ ID NO:4 |
| ATP5B-UF | 5′-GTGTCGTGGAGTCGGCAAGTGGGCGAAGCGGGAACCCGTGCACGGAAAATACAG | SEQ ID NO:5 |
| CETP-UF | 5′-GTGTCGTGGAGTCGGCAACGGAGCGATCACGTGGCAGATTACACCAAAGACTGTTTCCAA | SEQ ID NO:6 |
| E1F1A-UF | 5′-GTGTCGTGGAGTCGGCAAACGCGACGCACCTGCTCAAGATTGGCGGCATTGG | SEQ ID NO:7 |
| Cox6b-UF | 5′-GTGTCGTGGAGTCGGCAACCTCCCTCACGCGCTTGGGGCAGAGGGACTGGT | SEQ ID NO:8 |
| ATP5B-UR | 5′-ACCGACTCCAGCTCCCGAACCCTGTGAAGACCTCAGCAACCT | SEQ ID NO:9 |
| CETP-UR | 5′-ACCGACTCCAGCTCCCGAACTGACTGCAGGAAGCTCTGGAT | SEQ ID NO:10 |
| EIF1A-UR | 5′-ACCGACTCCAGCTCCCGAACCCCGGCCGCAGGAT | SEQ ID NO:11 |
| Cox6b-UR | 5′-ACCGACTCCAGCTCCCGAACACCGCTAAAGGAGGCGATATC | SEQ ID NO:12 |
| Uni1PCR37-UR | 5′-ACCGACTCCAGCTCCCGAAC | SEQ ID NO:13 |
| Un12PCR38-UF | 5′-GTGTCGTGGAGTCGGCAA | SEQ ID NO:14 |
| Code-1-FAM | FAM-5′-GTGGGCGAAGCGGGAA-MGB | SEQ ID NO:15 |
| Code-2-Vic | Vic-5′-CGGAGCGATCACGTGG-MGB | SEQ ID NO:16 |
| Code-2-FAM | FAM-5′-CGGAGCGATCACGTGG-MGB | SEQ ID NO:17 |
| Code-3-Ned | Ned-5′-ACGCGACGCACCTGCT-MGB | SEQ ID NO:18 |
| Code-3-FAM | FAM-5′-ACGCGACGCACCTGCT-MGB | SEQ ID NO:19 |
| Code-4-ROX | ROX-5′-CCTCCCTCACGCGCCT-MGB | SEQ ID NO:20 |
| Code-4-FAM | FAM-5′-CCTCCCTCACGCGCCT-MGB | SEQ ID NO:21 |
| Temp+ | 5′-GTGTCGTGGAGTCGGCAAGTGGGCGAAGCGGGAACCCGTGCACGGAAAATACAGAGGTTGCT | SEQ ID NO:22 |
| | GAGGTCTTCACAGGGTTCGGGAGCTGGAGTCGGT-3′ | |
| Temp− | 3′-CACAGCACCTCAGCCGTTCACCCGCTTCGCCCTTGGGCACGTGCCTTTTATGTCTCCAACGA | SEQ ID NO:23 |
| | CTCCAGAAGTGTCCCAAGCCCTCGACCTCAGCCA-5′ | |
| ATP5B-RP | 5′-CCTGTGAAGACCTCAGCAACCT | SEQ ID NO:24 |
| ATP5B-FAM(+) | FAM-5′-TTCTTGTCTCAGCCAT-MGB | SEQ ID NO:25 |

What is claimed is:

1. A method of obtaining a Cₑ value of a nucleic acid amplification reaction, comprising:
a) exponentially and linearly amplifying a target sequence in a coupled reaction under conditions in which the exponential amplification terminates before reaching a plateau and a reporter molecule generates a detectable signal proportional to the number of linear stranded amplicons;
b) measuring the detectable signal as a function of cycle number; and
c) obtaining therefrom the Cₑ value of the amplification reaction.

2. The method according to claim 1, wherein said conditions terminate said exponential amplification at or about the cycle number that said exponential amplification is capable of producing a detectable signal.

3. The method according to claim 1, wherein said conditions terminate said exponential amplification before the cycle number that said exponential amplification is capable of producing a detectable signal.

4. The method according to claim 1, wherein said reporter molecule is a hydrolyzable probe and said target sequence is amplified in a thermal cycling reaction comprising forward and reverse amplification primers, said hydrolyzable probe, and a thermostable polymerase having 5′-3′ nuclease activity, wherein said forward primer is in excess of said reverse primer, and said probe hybridizes to said target sequence 3′ relative to said forward primer, and wherein the conditions of said reaction are effective for said forward primer, said probe, and said target sequence to form a substrate for said nuclease activity and for said nuclease activity to hydrolyze said probe to generate said detectable signal.

5. The method according to claim 4, wherein said detectable signal is a fluorescence signal and said hydrolyzable probe is a self-quenching fluorescence probe.

6. The method according to claim 4, wherein said detectable signal is a fluorescence signal and said hydrolyzable probe comprises a 3′ and a 5′ sequence, wherein said 3′ sequence is suitable for hybridizing to said target sequence 3′ relative to said forward primer and said 5′ sequence is a cleavage sequence that is not suitable for hybridizing to said target sequence and comprises a fluorescent moiety, and wherein said nuclease activity releases said cleavage sequence, and said fluorescence signal is generated during capillary electrophoresis of the released cleavage sequence.

7. The method according to claim 4, where the Tₘs of said forward primer, said reverse primer, and said probe with said target sequence are within a range of about 5° C. or less.

8. The method according to claim 4, wherein said forward primer is in excess of said reverse primer by at least about 50:1.

9. The method according to claim 4, wherein the concentration of said reverse primer is about 10 to about 30 nM and the concentration of said forward primer is at least about 500 nM.

10. A method of obtaining the copy number of a target sequence, comprising:
a) amplifying a target sequence in a reaction that couples a linear phase with an exponential phase under conditions in which a reporter molecule generates a detectable signal proportional to the amount of linear phase amplicon generated, and in which the exponential phase terminates before it plateaus;
b) measuring the detectable signal as a function of cycle number;
c) obtaining therefrom the Cₑ value of the amplification reaction; and
d) obtaining from said Cₑ value the copy number of said target sequence.

11. The method according to claim 10, wherein said conditions terminate said exponential phase at or about the cycle number that said exponential phase is capable of producing a detectable signal.

12. The method according to claim 10, wherein said conditions terminate said exponential phase before the cycle number that said exponential phase is capable of producing a detectable signal.

13. The method according to claim 10, wherein said reporter molecule is a hydrolyzable probe and said target sequence is amplified in a thermal cycling reaction comprising forward and reverse amplification primers, said hydrolyzable probe, and a thermostable polymerase having 5′-3′ nuclease activity, wherein said forward primer is in excess of said reverse primer, and said probe hybridizes to said target sequence 3′ relative to said forward primer, and wherein the conditions of said reaction are effective for said forward primer, said probe, and said target sequence to form a substrate for said nuclease activity and for said nuclease activity to hydrolyze said probe to generate said detectable signal.

14. The method according to claim 13, wherein said detectable signal is a fluorescence signal and said hydrolyzable probe is a 5′-nuclease probe.

15. The method according to claim 13, wherein said detectable signal is a fluorescence signal and said hydrolyzable probe comprises a 3′ and a 5′ sequence, wherein said 3′ sequence is suitable for hybridizing to said target sequence 3′ relative to said forward primer and said 5′ sequence is a cleavage sequence that is not suitable for hybridizing to said target sequence and comprises a fluorescent moiety, and wherein said nuclease activity releases said cleavage sequence, and said fluorescence signal is generated during capillary electrophoresis of the released cleavage sequence.

16. The method according to claim 13, where the Tₘs of said forward primer, said reverse primer, and said probe with said target sequence are within a range of about 5° C. or less.

17. The method according to claim 10, wherein said reporter molecule is a PNA probe comprising a sequence substantially complementary to said linear amplicons and a system suitable for producing a detectable signal when said PNA probe is hybridized to said linear amplicons.

18. The method according to claim 13, wherein said forward primer is in excess of said reverse primer by at least about 50:1.

19. The method according to claim 13, wherein the concentration of said reverse primer is about 10 to about 30 nM and the concentration of said forward primer is at least about 500 nM.

20. A computer readable memory to direct a computer to function in a specified manner, comprising:
executable instructions to direct a computer to obtain the value assigned to a fluorescent signal measured at a user-selected cycle number of an amplification reaction, wherein said reaction couples exponential and linear amplification of a target sequence to produce double-and single stranded amplicons, wherein the conditions of said reaction are effective to terminate said exponential amplification before the production of double-stranded amplicons plateaus, to produce linear amplicons at a rate proportional to the number of double-stranded amplicons, and to produce a detectable signal proportional to said cycle number;
executable instructions to calculate the Cₑ value of said exponential amplification reaction from a first-order kinetic relationship of the measured signal, said cycle number, the rate of linear amplification, and the minimum number of double-stranded amplicons capable of being measured by said detector; and
executable instructions to calculate from said Cₑ value the copy number of said target sequence.

21. The computer readable memory according to claim 20, wherein said conditions are effective to terminate said exponential amplification when the production of double-stranded target amplicons is at or about the minimum number of double-stranded amplicons capable of being detected by said system.

22. The computer readable memory according to claim 20, wherein said conditions are effective to terminate said exponential amplification before the production of double-stranded target amplicons is at or about the minimum number of double-stranded amplicons capable of being detected by said system.

23. The computer readable memory according to claim 20, wherein the minimum number of double-stranded amplicons capable of being detected by said system is determined by the exponential amplification of a control sequence and monitoring is determined by the exponential amplification of a control sequence and monitoring double-stranded amplicon production using said system.

24. The computer readable memory according to claim 20, wherein the minimum number of double-stranded amplicons capable of being detected by said system is obtained from a set of standard reactions.

25. The method according to claim 20, wherein the minimum number of double-stranded amplicons capable of being detected by said system is a constant.

26. A method of quantitating one or more target sequences comprising:
a) exponentially amplifying one or more target sequences in a reaction that terminates when a selected number of exponential amplicons are produced from each target sequence;
b) linearly amplifying each exponential amplicon to produce linear amplicons in a coupled reaction that produces a detectable signal proportional to each linear amplicon;
c) measuring said detectable signals as a function of amplification cycle number; and
d) determining therefrom the quantity of said one or more target sequences.

27. The method according to claim 26, wherein the exponential reaction is a polymerization reaction.

28. The method according to claim 26, wherein the linear reaction is a polymerization reaction.

29. The method according to claim 26, wherein said detectable signal is produced by a reporter molecule.

30. The method according to claim 29, wherein said reporter molecule is a self-quenching fluorescence probe.

31. The method according to claim 29, wherein said reporter molecule is a PNA probe.

32. The method according to claim 29, wherein said reporter molecule is a hydrolyzable probe.

33. The method according to claim 32, wherein said hydrolyzable probe is a 5′ nuclease probe.

34. The method according to claim 32, wherein said hydrolyzable probe is a “flap” probe.


SEQUENCE LISTING (25 sequences)

SEQ ID NO: 1 - DNA, 16 bp, Homo sapiens
atggctgagacaagaa

SEQ ID NO: 2 - DNA, 16 bp, Homo sapiens
agctgctctcagtcaa

SEQ ID NO: 3 - DNA, 14 bp, Homo sapiens
cacccggcccacgg

SEQ ID NO: 4 - DNA, 16 bp, Homo sapiens
acacgctggtaccatt

SEQ ID NO: 5 - DNA, 54 bp, Artificial Sequence
  Description of Artificial Sequence Synthetic oligonucleotide
gtgtcgtggagtcggcaagtgggcgaagcgggaacccgtgcacggaaaatacag

SEQ ID NO: 6 - DNA, 60 bp, Artificial Sequence
  Description of Artificial Sequence Synthetic oligonucleotide
gtgtcgtggagtcggcaacggagcgatcacgtggcagattacaccaaagactgtttccaa

SEQ ID NO: 7 - DNA, 52 bp, Artificial Sequence
  Description of Artificial Sequence Synthetic oligonucleotide
gtgtcgtggagtcggcaaacgcgacgcacctgctcaagattggcggcattgg

SEQ ID NO: 8 - DNA, 51 bp, Artificial Sequence
  Description of Artificial Sequence Synthetic oligonucleotide
gtgtcgtggagtcggcaacctccctcacgcgcttggggcagagggactggt

SEQ ID NO: 9 - DNA, 42 bp, Artificial Sequence
  Description of Artificial Sequence Synthetic oligonucleotide
accgactccagctcccgaaccctgtgaagacctcagcaacct

SEQ ID NO: 10 - DNA, 41 bp, Artificial Sequence
  Description of Artificial Sequence Synthetic oligonucleotide
accgactccagctcccgaactgactgcaggaagctctggat

SEQ ID NO: 11 - DNA, 34 bp, Artificial Sequence
  Description of Artificial Sequence Synthetic oligonucleotide
accgactccagctcccgaaccccggccgcaggat

SEQ ID NO: 12 - DNA, 41 bp, Artificial Sequence
  Description of Artificial Sequence Synthetic oligonucleotide
accgactccagctcccgaacaccgctaaaggaggcgatatc

SEQ ID NO: 13 - DNA, 20 bp, Artificial Sequence
  Description of Artificial Sequence Synthetic oligonucleotide
accgactccagctcccgaac

SEQ ID NO: 14 - DNA, 18 bp, Artificial Sequence
  Description of Artificial Sequence Synthetic oligonucleotide
gtgtcgtggagtcggcaa

SEQ ID NO: 15 - DNA, 16 bp, Artificial Sequence
  Description of Artificial Sequence Synthetic oligonucleotide
gtgggcgaagcgggaa

SEQ ID NO: 16 - DNA, 16 bp, Artificial Sequence
  Description of Artificial Sequence Synthetic oligonucleotide
cggagcgatcacgtgg

SEQ ID NO: 17 - DNA, 16 bp, Artificial Sequence
  Description of Artificial Sequence Synthetic oligonucleotide
cggagcgatcacgtgg

SEQ ID NO: 18 - DNA, 16 bp, Artificial Sequence
  Description of Artificial Sequence Synthetic oligonucleotide
acgcgacgcacctgct

SEQ ID NO: 19 - DNA, 16 bp, Artificial Sequence
  Description of Artificial Sequence Synthetic oligonucleotide
acgcgacgcacctgct

SEQ ID NO: 20 - DNA, 16 bp, Artificial Sequence
  Description of Artificial Sequence Synthetic oligonucleotide
cctccctcacgcgcct

SEQ ID NO: 21 - DNA, 16 bp, Artificial Sequence
  Description of Artificial Sequence Synthetic oligonucleotide
cctccctcacgcgcct

SEQ ID NO: 22 - DNA, 96 bp, Artificial Sequence
  Description of Artificial Sequence Synthetic oligonucleotide
gtgtcgtggagtcggcaagtgggcgaagcgggaacccgtgcacggaaaatacagaggttgctgaggtcttcacagggttcgggagctggagtcggt

SEQ ID NO: 23 - DNA, 96 bp, Artificial Sequence
  Description of Artificial Sequence Synthetic oligonucleotide
cacagcacctcagccgttcacccgcttcgcccttgggcacgtgccttttatgtctccaacgactccagaagtgtcccaagccctcgacctcagcca

SEQ ID NO: 24 - DNA, 22 bp, Artificial Sequence
  Description of Artificial Sequence Synthetic oligonucleotide
cctgtgaagacctcagcaacct

SEQ ID NO: 25 - DNA, 16 bp, Artificial Sequence
  Description of Artificial Sequence Synthetic oligonucleotide
ttcttgtctcagccat